(19) United States
(12) Patent Application Publication
(10) Pub. No.: US 2025/0122468 A1
(43) Pub. Date: Apr. 17, 2025

(54) BIOINK FOR REPRODUCIBLE PRODUCTION OF 3D TUMOR TISSUE SCAFFOLDS

(71) Applicant: ISCAFF PHARMA AB, Göteborg (SE)

(72) Inventors: Göran LANDBERG, Halmstad (SE); Anders STÅHLBERG, Kållered (SE)

(21) Appl. No.: 18/568,488

(22) PCT Filed: Jun. 9, 2022
(86) PCT No.: PCT/SE2022/050564

§ 371 Date: Dec. 8, 2023

(30) Foreign Application Priority Data
Jun. 10, 2021 (SE) 2150734-8

(51) Int. Cl. C12N 5/00; B33Y 10/00; B33Y 70/00; B33Y 80/00; G01N 33/68

(52) U.S. Cl. CPC C12N 5/0068; B33Y 10/00; B33Y 70/00; B33Y 80/00; G01N 33/6848; C12N 2513/00; C12N 2533/54

(57) ABSTRACT

The present invention relies on the discovery that certain proteins are enriched in the tumor microenvironment of different types of tumors and subgroups of tumors but not in others and that such proteins can be used as components of bioinks. The bioinks can be used in production of reproducible tumor type-specific 3D scaffolds to provide tumor type-specific in vitro models for various cancer research applications.

### TECHNICAL FIELD

The present invention generally relates to the field of bioinks and the use thereof for production of three-dimensional (3D) scaffolds, and in particular to identification of protein components for such bioinks to reproducibly produce 3D scaffolds useful as in vitro models in cancer research applications.

### BACKGROUND OF THE INVENTION

Cancer is a heterogeneous disease where the extracellular microenvironment of a tumor, or tumor microenvironment for short, including extracellular components, plays a crucial role in tumor progression, potentially modulating treatment response. The extracellular microenvironment plays an important role in structural and functional organization of the tumor. Different approaches have been used to develop 3D models able to recapitulate the complexity of the extracellular microenvironment of tumor tissue. The mechano-chemical stimulation and biological interaction between cells and the extracellular microenvironment influence proliferation, survival, migration, invasiveness, and drug response. The tumor microenvironment also influences survival of cancer stem cells (CSC), a population that displays stem cell properties, such as self-renewal, multipotent differentiation, and a high tumor-initiating capacity. The CSC phenotype has been linked to poor prognostic features, drug resistance and increased risk of disease recurrences.

Traditional two-dimensional (2D) culture systems used to study cancer lack the complexity of 3D cell-to-cell contacts and interactions between cells and the extracellular microenvironment that occur in vivo. The use of scaffolds is an emerging approach to generate 3D systems that are able to mimic the in vivo tumor microenvironment characteristics. 3D scaffolds constructed with different fiber diameters, fiber arrangements, pore sizes, and/or pore structures can be prepared by various techniques, including electrospinning, wet spinning, melt spinning, 3D printing, pouring, phase separation, and particle leaching. 3D scaffolds can be produced using biocompatible and biodegradable synthetic materials like polymers, or using materials derived from natural sources, such as fibrous proteins, or biopolymers. Nevertheless, the selection of the components of the material is crucial due to several factors related to the scaffold composition and structure, such as stiffness, porosity, dimensionality, and presence of specific proteins, all influencing the behaviors of the attached and growing cancer cells

WO 2019/122351 discloses cellulose nanofibril-based bioinks comprising human tissue-specific extracellular matrix (ECM) obtained from decellularized tissue. In more detail, human decellularized ECM is lyophilized and milled into ECM powder that is combined with a bioink comprising nanofibrilated cellulose (NFC) and sodium alginate.

The complex structure of the tumor microenvironment has, however, been shown to provide more than just structural support for cancer cells, and it can also influence tumor progression, treatment resistance, as well as metastasis. Thus, the tumor microenvironment provides an underestimated diagnostic parameter in understanding disease progression. In recent years, the importance of the tumor microenvironment has gained increasing interest, but many questions remain and there is still an unmet need to identify specific markers or specific combinations of markers in the tumor microenvironment that can be used to better diagnose patients and potentially provide novel targets for anti-cancer therapies. Being able to deduce diagnostic patterns that are unique to specific cancer states is challenging because of the biological variability in an individual patient's sample or samples, the variability between patients, and because of the range of biomarker concentrations when patients are compared. Therefore, standardization issues regarding biological variation, preanalytical variables, and analytical variability must be tackled before standard values can be established.

There is thus still a need for improvement within the field of cancer diagnosis and treatment, in particular regarding reproducible in vitro assays to study the influence of the tumor microenvironment on the cancer cells and for replicating tumors with specific properties.

### SUMMARY OF THE INVENTION

It is a general objective to use patient-derived decellularized tumor tissue to identify and select proteins as components in a bioink for production of reproducible 3D scaffolds that can be used as in vitro models in cancer research applications.

It is a particular objective to produce bioinks for production of reproducible 3D scaffolds mimicking tumor tissue-specific microenvironments.

These and other objectives are met by embodiments as disclosed herein.

The present invention is defined in the independent claims. Further embodiments of the invention are defined in the dependent claims.

An aspect of the invention relates to a method for producing a bioink. The method comprises analyzing a protein composition of a decellularized tumor tissue or tissues obtained from at least one tumor characterized by at least one defined tumor property or from at least one tumor of a respective patient characterized by at least one defined patient property. The method also comprises selecting at least one protein that is differentially expressed in the decellularized tumor tissue or tissues as compared to a reference tissue or tissues. The method further comprises producing a bioink comprising the selected at least one protein, or a domain thereof, and configured for production of a 3D scaffold mimicking a tumor characterized by the at least one defined tumor property or a tumor of a patient characterized by the at least one defined patient property.

The present invention also relates to a method for producing a 3D scaffold. The method comprises producing a bioink according to above and producing the 3D scaffold using the bioink.

Aspects of the invention also relate to a bioink obtainable by the method above and a bioink comprising at least one protein, or a domain thereof, which is differentially expressed in a decellularized tumor tissue as compared to a reference tissue.

A further aspect of the invention relates to a 3D scaffold for cell culturing obtainable from the bioink according to above.

The present invention also relates to a cell culturing method comprising adding cells to a 3D scaffold according to above and culturing the cells in the 3D scaffold.

A bioink as produced according to the present invention has a protein composition selected to mimic specific characteristics of a tumor. This means that 3D scaffolds produced using the bioink by, for instance bioprinting and/or electrospinning, will present a microenvironment to cells cultured in the 3D scaffolds that mimics the particular in vivo microenvironment of a tumor with its specific characteristics and is capable of inducing a specific gene expression in the cancer cells cultured in the 3D scaffold. The bioink of the invention further has a well-defined composition including selected proteins that enables reproducible bioprinting of the 3D scaffolds. The 3D scaffolds obtainable according to the invention can thereby be used as in vitro models or tools in cancer research applications to mimic a defined in vivo tumor microenvironment.

### BRIEF DESCRIPTION OF DRAWINGS

The embodiments, together with further objects and advantages thereof, may best be understood by making reference to the following description taken together with the accompanying drawings, in which:

FIG. 1. Schematic description of the workflow from tissue collection to data analysis.

FIG. 2. Protein classification according to PANTHER Protein Class. Pie chart illustrating percentage of types of proteins in the 1844 proteins detected in patient-derived decellularized tumor samples to each category against 1158 total number of Protein Class hits resulting from PANTHER classification analysis. Noteworthy is that extracellular matrix proteins only represent about 4% of the total number of proteins.

FIG. 3. Top 15 proteins showing the highest average protein contents in all analyzed patient-derived decellularized tumor samples. The Search Tool for the Retrieval of Interacting Genes Database (STRING) (https://www.strin g-db.org/) was used to illustrate known or predicted protein-protein interactions (lines) for the top 15 proteins.

FIG. 4. Top 50 proteins in patient-derived decellularized tumor samples associated with recurrence and top 24 proteins in patient-derived decellularized tumor samples associated with no recurrence. A) Heatmap representation of proteins significantly and differently expressed between patient-derived scaffolds from tumors derived from patients with or without disease recurrence. B-C) Patient-derived scaffold protein classification according to PANTHER Protein Class. The pie charts illustrate the percentage of the various functional classification from patients with (B) recurrence or (C) no recurrence.

FIG. 5. Patient-derived decellularized tumor samples from tumors with high and low tumor grade can be subdivided based on their extracellular matrix composition. Self-organizing map (SOM) analyses (GenEx, multid software) formed four groups of the 65 patient-derived decellularized tumor samples (PDSs) and 8 PDSs from adjacent tissues using the identified 126 extracellular matrix proteins (ECM as illustrated in a heat-map). The distribution of the SOM groups in relation to estrogen receptor (ER) and progesterone receptor (PR) status, size, high-grade, lymph node-metastasis and patients characteristics as disease-recurrence and age is also presented.

FIG. 6. Extracellular matrix proteins differing between low and high-grade tumors. Top 10 proteins identified with dynamic principle component analysis from the 126 extracellular matrix proteins in the patient-derived decellularized tumor samples defining high-grade tumors from low-grade. (*p≤0.05, ** p≤0.01, *** p≤0.001, Mann-Whitney U-test).

FIG. 7. Collagen abundance in the breast cancer microenvironment decreases with patient's age. In 10 out of 20 collagens patient-derived decellularized tumor samples collagen expression could be stratified based on patient's age. (*p≤0.05, ** p≤0.01, *** p≤0.001, Two-Way ANOVA with Tukey's multiple comparison test).

FIG. 8. Comparison of protein family distribution using PANTHER Protein classifications among proteins differentially expressed in high grade (III, 514 proteins) versus lower grade (I-II, 117) breast cancer using patient-derived decellularized tumor samples.

FIG. 9. ECM proteins and defense/immunity proteins are more often present in lobular cancer compared to in ductal breast cancer. Comparison of protein family distribution using PANTHER Protein classifications among proteins differentially expressed in lobular cancer (138 proteins) versus ductal breast cancer (324) breast cancer using patient-derived decellularized tumor samples.

FIG. 10. 73 of 74 proteins differing between recurrent and not recurrent breast cancer (FIG. 3) using patient-derived decellularized breast cancer samples are also present in varying amounts in patient-derived decellularized tumor samples from colon cancer and ovarian cancer. Selected proteins are presented in detail in histograms and the average expression of all proteins in the different cancer forms as heatmaps.

FIG. 11. Out of 74 differentially expressed proteins in patient-derived decellularized tissues from breast cancer with or without recurrences, 7 were identified as keratins. As illustrated in the figure using Log 2 expression of proteins in the keratin family, several keratins showed significantly higher protein contents in recurrent diseases. Boxplots illustrates median, quartiles and min/max range, Student's t-test. *≤0.05, **≤0.001.

FIG. 12. Lacritin is expressed at higher levels in patient-derived decellularized tumor samples associated with disease recurrence compared to patient-derived decellularized tumor samples associated with no recurrence. (NT=normal tissue). A Kaplan Meier plot is further illustrating disease recurrence and time after surgery divided according to Lacritin protein expression and median values.

FIG. 13. Cancer cells were cultured in decellularized patient-derived scaffolds (PDS), in matrigel scaffolds, in 3D printed alginate scaffolds, and in electrospun nanofiber 3D scaffolds. Analysis of 20 genes linked to cancer-associated molecular processes in the cancer cells revealed that the cancer cell gene expression depends on the type of 3D scaffold in which the cells are cultured, where electrospun nanofibers induced a cancer cell gene expression more similar to the gene expression induced by PDS scaffolds as compared to the gene expression induced by matrigel or by 3D-printed alginate scaffolds.

FIG. 14. Cancer cells were cultured in alginate-based 3D printed scaffolds where keratin 10 (KRT10) had previously been linked to the alginate. Scaffolds were printed using the alginate-KR10 diluted into two different concentrations (25% alginate-KR10 and 50% alginate-KR10). Scaffolds made by 3D printed alginate without KRT10 as well as cell-free PDSs were used as controls. 20 selected genes, indicative of important molecular tumor processes, were analyzed and the results clearly show that the modification of the alginate with KRT10 has a biological effect. Values presented in the figure are normalized to the same genes expressed by cancer cells grown in conventional 2D cultures.

FIG. 15. Protein and PDS clustering data obtained with K-means cluster analysis of ECM proteins in relation to all proteins detected in cell-free PDSs. This analysis illustrates how various proteins present in the scaffolds affect the positioning and clustering of PDSs (A) and the positioning and clustering of certain groups of proteins (B). ECM proteins (black dots mostly cluster to the left) and are clearly influencing the positioning of the various PDSs in a different way as compared to the bulk proteins (grey dots).

### DEFINITIONS

“Domain” of a protein as used herein relates to a domain or portion of a protein capable of exerting the desired function of the full protein in a 3D scaffold produced, such as bioprinted, from a bioink comprising the domain of the protein. The domain of the protein could be a “structural domain” for proteins exerting a structural function in an ECM or a microenvironment of a tumor tissue. Such a structural domain may then exert structural support in the 3D scaffold. An example of a structural domain in the case of a membrane protein is the extracellular domain or portion of the membrane protein. Another example of structural domain is a portion of a protein interconnecting other molecules, such as proteins, in an ECM or microenvironment of a tumor tissue. A domain of a protein could also be a “functional domain” of an enzyme. In such a case, the functional domain is capable of catalyzing the same chemical reaction as the full enzyme or is capable of exerting a substantially same enzymatic reaction when the functional domain is in an isolated form as the functional domain exerts when included in the full protein or enzyme. The functional domain thereby corresponds to an enzymatically active portion of the enzyme. A further example of a domain is a “binding domain” or antigenic portion of a protein. In such a case, the domain corresponds to the portion of the protein to which another molecule, such as receptor, antibody or other protein, binds. Furthermore, many proteins consist of several distinct protein domains or structural units that fold more or less independently of each other. In an embodiment, the domain of the protein corresponds to at least 10 amino acids, preferably at least 10 consecutive amino acids, preferably at least 15 (consecutive) amino acids, such as at least 20 (consecutive) amino acids, or at least 25 (consecutive) amino acids, of the protein, preferably at least 30 (consecutive) amino acids, of the protein, such as at least 35 (consecutive) amino acids, at least 40 (consecutive) amino acids, at least 45 (consecutive) amino acids, or at least 50 (consecutive) amino acids, i.e., at least 50 amino acids, preferably at least 50 consecutive amino acids, of the protein. The domain may also comprise a larger number of amino acids, preferably consecutive amino acids, of the protein, such as at least 55, at least 60, at least 65, at least 70, at least 75, at least 80, at least 85, at least 90, at least 95, or at least 100 amino acids, preferably consecutive amino acids, of the protein, depending on the total number of amino acids in the protein. In another embodiment, the domain of the protein corresponds to at least 25% of the amino acid sequence of the full protein, preferably at least 30%, at least 35%, at least 40%, at least 45% or at least 50% of the amino acid sequence. The domain may correspond to even larger parts of the amino acid sequence of the protein, such as at least 55%, at least 60%, at least 65%, at least 70%, at least 75%, at least 80%, at least 85%, at least 90% or at least 95% of the amino acid sequence of the protein, or even more such as at least 96%, at least 97%, at least 98% or at least 99%. The amino acid sequence of the domain of the protein does not necessarily have to correspond to a consecutive portion of the amino acid sequence of the protein. For instance, the domain could correspond to N-terminal and C-terminal portions of the protein fused together to form the domain but excluding the amino acid sequence of an intermediate portion of the protein.

“ECM proteins” as used herein are proteins and polypeptides providing structural support of the extracellular matrix (EMC), which is a 3D network consisting of extracellular macromolecules, such as collagens, laminins, and proteoglycans, fibrous proteins and minerals, that provide structural and biochemical support to surrounding cells. The extracellular microenvironment of a tumor (tumor microenvironment) comprises such ECM proteins but also a large amount of non-ECM proteins as disclosed herein. ECM proteins are, in a particular embodiment, proteins classified as ECM proteins using pathway analyzing and classifying programs, such as PANTHER or GoMiner.

Gene names and corresponding protein names referred to in this document and shown in the drawings are listed in Annex A.

### DETAILED DESCRIPTION OF INVENTION

The present invention generally relates to the field of bioinks and three-dimensional (3D) scaffolds produced using the bioinks, and in particular to identification of protein components for such bioinks to reproducibly produce 3D scaffolds useful as in vitro models in cancer research applications.

By removing tumor cells from a tumor tissue sample, a patient-derived cell-free (decellularized) tumor tissue is obtained. Such a decellularized tumor tissue maintains the microenvironment of the particular tumor tissue, from which it is obtained, and thereby comprises components present in vivo in the tumor tissue. This microenvironment of a tumor tissue consists not only of structural extracellular matrix (ECM) proteins, but also numerous other components derived from the cells that reside in, or enter, the tumor tissue, such as the cancer cells, fibroblasts, endothelial cells forming the blood vessels of the tumor, and immune cells. Both ECM components and the numerous other components within the tumor microenvironment may, individually or in combination, have the ability to influence how cancer cells behave within their environment. In other words, these molecules give rise to different and varying tumor properties depending on, for instance, which component or combination of components that is present, and also the relative levels (ratio) between certain components within the microenvironment.

However, components within the tumor microenvironment vary between cancer types, between tumors of the same cancer type, between individuals and even within the same individual under different stimuli, in different parts of the tumor, or in different cancer disease stages. This means that bioinks comprising, for instance, ECM components as produced by homogenizing decellularized tissue will not have consistent and well-defined components as the ECM components will vary from tissue to tissue and even within one and the same tissue over time. This means that 3D scaffolds produced from such bioinks comprising homogenized decellularized tumor tissue will not be consistent due to varying bioink compositions.

Furthermore, producing 3D scaffolds from bioinks with substantially only ECM components might enable production of 3D scaffolds that reflect the physical structure of a tumor microenvironment. However, a 3D scaffold produced from merely such ECM components will not correctly reflect the entire tumor microenvironment and its characteristics. Hence, such a 3D scaffold will not correctly reflect the tumor properties of a real tumor in a patient. In addition, producing 3D scaffolds from bioinks with just any ECM components, for instance ECM components selected solely for structural purposes, may actually affect the behavior of the cancer cells in an unexpected and possibly also in an undesired way. As an example, cancer cells cultured in such a 3D scaffold may no longer be suitable as in vitro models or tools in cancer research mimics as they are not representative of the in vivo tumor microenvironment of a tumor with defined properties.

The present invention in clear contrast enables reproducible production of 3D scaffolds produced from bioinks having well-defined and consistent compositions. Such 3D scaffolds mimic a tumor characterized by at least one defined tumor property or a tumor of a patient characterized by at least one defined patient property and the cancer cells grown in such 3D scaffolds will behave in a predicted manner. As such, the components of the bioinks have been selected based on analysis of patient-derived decellularized tumor tissues and their components to thereby enable production of 3D scaffolds mimicking selected characteristics or properties of the tumor tissues. As illustrative examples, decellularized tumor tissues derived from tumors of different grades or malignancies have differently expressed protein components. The components of the bioink can then be selected and used at a specific or a relative amount, to achieve a desired tumor grade or malignancy.

The current invention is based on the detailed analysis of the composition of decellularized tumor tissues using mass spectrometry. The analysis identified protein markers reflecting tumor properties and such protein markers can thereby be included individually or in different combinations in bioinks for specific and reproducible production of 3D scaffolds that can be used as in vitro models to better diagnose patients and to provide novel targets for anti-cancer therapies.

Patient-derived cell-free or decellularized tumor tissue samples are derived from tumors in patients, and they provide information about the particular tumor microenvironment. Proteins in the microenvironment defined by the decellularized tumor tissue are identified and selected as components of bioinks for production of 3D scaffolds for culturing of cancer cells and other cell types.

The complexity of the in vivo tumor microenvironment and its effects on tumor properties, such as tumor growth and susceptibility to cancer treatment, including immunotherapy, differs to most model systems used in cancer research today. The in vitro models used today are typically represented by cell cultures of cancer cell lines growing on plastics under high oxygen supply and immense growth factor activation. The in vivo animal models, using mainly immunocompromised mice, at least in part create more in vivo like cancer growth conditions by the use of implanted human tumors in the form of xenografts. Compared to the in vitro models, such in vivo model systems can be used for drug testing and studies of cancer growth in a more complex environment, but they have several limitations associated with the immunocompromised mice as well as non-human stromal reactions. For instance, breast cancer cell growth in xenografts does not mimic in vivo growth in patients as the cells tend to be less infiltrative and also to have large central necrotic areas due to rapid cell division in relation to angiogenic support. This creates an artificial cancer growth system that might be superior to less complex cell cultures but is still not close enough to real in vivo conditions.

In contrast to these animal models, patient-derived decellularized tumor tissue can be used as in vitro models to better mimic in vivo tumor conditions. However, although the patient-derived decellularized tumor tissue is able to mimic in vivo tumor conditions, such tissue is very limited in supply. The bioink as produced according to the invention can then be used to produce 3D scaffolds that can be used instead of patient-derived decellularized tumor tissue as reproducible 3D models for cancer research applications.

The tumor microenvironment is known for its complexity, both in its content as well as in its dynamic nature, which is difficult to study using two-dimensional (2D) cell culture models. Several advances in tissue engineering have allowed more physiologically relevant 3D in vitro cancer or tumor models, such as spheroid cultures, biopolymer scaffolds, and cancer-on-a-chip devices. Bioprinting 3D scaffolds using various ECM proteins has also been described. However, although these models serve as powerful tools for dissecting the roles of various biochemical and biophysical cues in carcinoma initiation and progression, they lack the ability to mimic the tumor microenvironment of a certain tumor and its specific tumor properties, and they also lack the ability to mimic the tumor environment from a patient having certain characteristics (such as age). By virtue of its ability both to mimic the microenvironment of a tumor with selected tumor properties and to, in a reproducible way, precisely define perfusable networks and position of various cell types in a high-throughput manner, preparing 3D scaffolds using bioinks of the invention has the potential to more closely recapitulate the cancer microenvironment, relative to current methods.

In addition, combining the bioink of the current invention with specific production technologies for 3D scaffold production can be used to optimize desired tumor properties of the produced 3D scaffolds. As an example, different production technologies will result in 3D scaffolds with various physical properties in terms of, for instance, fiber diameter, fiber arrangement, pore size, and pore structures even when the 3D scaffolds are produced from the same bioink. Hence, not only the components of the bioink but also the production technology or method may be selected to achieve a 3D scaffold mimicking a tumor characterized by at least one defined tumor property or a tumor of a patient characterized by at least one defined patient property.

A bioink of the invention may, in an embodiment, comprise a set of one or more base components that are typically selected to produce the physical structure of the 3D scaffold, e.g., formation of fibers forming the 3D scaffold. According to the invention, at least one protein that is differentially expressed in decellularized tumor tissue(s) as compared to reference tissue(s) is selected and included in the bioink to enable production of a 3D scaffold mimicking a tumor characterized by at least one defined tumor property or a tumor of a patient characterized by at least one defined patient property. In an embodiment, the one or more base components of the bioink is or are preferably selected at least partly based on the production method or technology used to produce the 3D scaffold from the bioink, such as depending on whether the 3D scaffold is produced using electrospinning, wet spinning, melt spinning, 3D printing, pouring, phase separation, and/or particle leaching.

A decellularized tumor tissue as referred to herein is a cell-free tumor tissue obtained from a tumor in a subject or patient. The microenvironment of a tumor generally comprises a collection of molecules, including ECM proteins and several other types of proteins, secreted by cells that provide structural and/or biochemical support to the surrounding cells. The network of these molecules deposited into the tumor microenvironment constitutes a 3D matrix for cells in the tumor. Typically, the matrix provides a physical 3D structure for the tumor, but also a microenvironment for cells, with which the cells can interact. A 3D tumor matrix may comprise, for example, collagen and various tumor promoting factors, such as growth factors, as well as inhibitors affecting cancer cell behaviors. A decellularized tumor tissue comprises decellularized extracellular material obtained from the tumor, in which the original 3D structure is substantially preserved. The bioactivity of the decellularized tumor tissue is substantially preserved. A decellularized tumor tissue allows effective attachment, migration, proliferation and 3D organization of cells cultures therein. Generally, the decellularized tumor tissue is substantially free of cells, in particular cancer cells. This may be assessed by any suitable means. Merely by way of example, sectioning and microscopic visualization may be used to determine the presence or absence of nuclei, which are indicative of cells, or DNA analysis may be used. Substantially free of cells means that cells are not detectable in the assessments.

A sample comprising tumor tissue from a tumor may be prepared using methods known in the art from, for example, a biopsy. A decellularized tumor tissue may be obtained from a tumor using suitable decellularizing methods to remove cells, while preserving the basic tumor matrix composition. A suitable method is disclosed in Example 1.

An aspect of the invention relates to a method for producing a bioink. The method comprises analyzing protein composition of a decellularized tumor tissue or tissues obtained from at least one tumor characterized by at least one defined tumor property or from at least one tumor of a respective patient characterized by at least one defined patient property. The method also comprises selecting at least one protein that is differentially expressed in the decellularized tumor tissue or tissues as compared to a reference tissue or tissues. The method further comprises producing a bioink comprising the selected at least one protein or a domain thereof and configured for production of a 3D scaffold mimicking a tumor characterized by the at least one defined tumor property or a tumor of a patient characterized by the at least one defined patient property.

This aspect of the invention is based on experimental results as presented herein obtained from mass spectrometry (MS) analysis of protein compositions of decellularized tumor tissues. The MS analyses indicated that the protein compositions of the decellularized tumor tissues differed significantly between tumors characterized by different tumor properties and also between tumors derived from patients with different patient properties. Thus, the tumor microenvironment and its composition are dependent on tumor and/or patient properties. This aspect of the invention thereby identifies and selects individual proteins, groups of proteins, and/or protein families that are differently expressed in a target decellularized tumor tissue and include that protein(s), groups of proteins, or a domain(s) thereof, in the bioink. A 3D scaffold produced from such a bioink will then mimic a tumor characterized by the at least one tumor property or as obtained from a patient characterized by the at least one defined patient property. The present invention thereby enables production of tailored bioinks that can be used for reproducible production of 3D scaffolds mimicking selected tumor microenvironments.

Decellularized tumor tissue is obtained from one or more tumors having at least one desired tumor property, also referred to as clinical variable of the tumor or tumors herein.

Generally, tumors have properties characterizing the tumor including its malignancy, which is typically defined by tumor grades or histological grades; cancer type, which defines the cancer disease the tumor is causing, such as breast cancer type; tumor location, i.e., the tissue or organ from which the tumor originates; differentiation capability; proliferation capability; invasiveness, i.e., infiltration capacity; metastasizing capability; epithelial-mesenchymal transition (EMT) capability; and/or cancer stem cell (CSC) capability.

For instance, a tumor may be assigned a particular grade, with higher grade indicating a more aggressive tumor. Tumor grade is usually assigned according to the appearance of the tumor cells, for example under a microscope. Grading systems for tumors are known to the skilled person. Higher grade tumors are often linked to more aggressive clinical behaviors and cancer progression as compared to low grade tumors. Typically, a progressing tumor has one or more of increased invasiveness, higher malignancy grade or malignancy potential, increased risk of recurrence, increased resistance to treatment, and/or increased tumor proliferation, compared to a non-progressing tumor.

Generally, tumor property as used herein refers to any clinically relevant characteristics of a tumor. Tumor properties may be those associated with, or indicative of, a progressing tumor. Such tumor properties may be those which are significant in determining tumor progression, for example, properties which are useful for distinguishing progressing tumors from non-progressing tumors. Suitable tumor properties may include, for example, invasiveness, migration, malignancy grade or malignancy potential, risk of recurrence, resistance to treatment, and/or tumor proliferation.

Hence, in an embodiment, the tumor has at least one defined tumor property selected from the group consisting of tumor grade, tumor size, cancer type or subtype, tumor location, differentiation capability, proliferation capability, infiltration capability, metastasizing capability, EMT capability, CSC capability estrogen receptor (ER) status, progesterone receptor (PR) status and recurrence.

In a particular embodiment, the tumor has at least one defined tumor property selected from the group consisting of tumor grade, tumor size, cancer type or subtype, ER status, PR status and recurrence, preferably the at least one defined tumor property is selected from the group consisting of tumor grade, tumor size, ER status, PR status and recurrence.

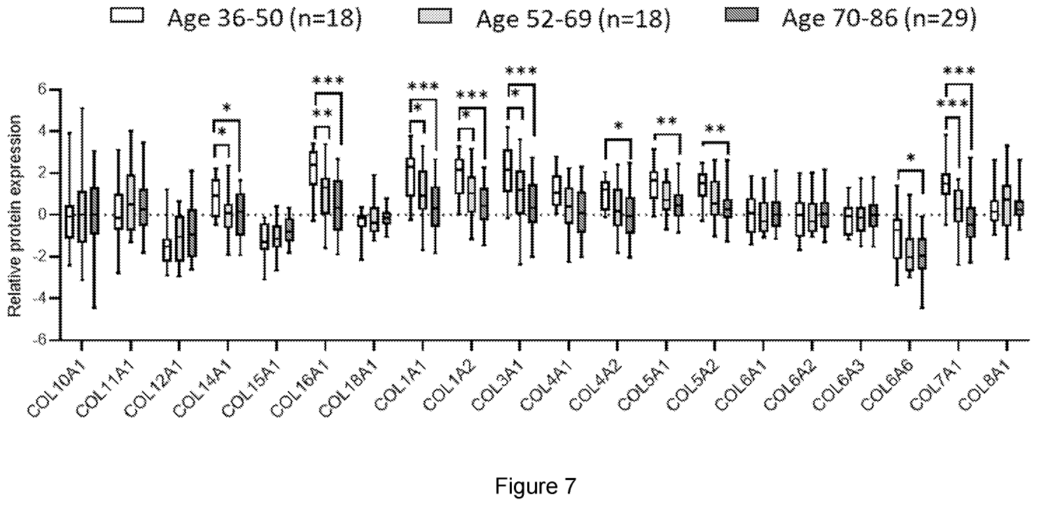

Decellularized tumor tissue or tissues is or are obtained from at least one tumor obtained from a respective patient having at least one desired patient property. An illustrative, but non-limiting, example of such a patient property is the age of the patient. For instance, MS analyses of decellularized tumor tissue from patients of different ages indicated that the protein composition of the decellularized tumor tissue was correlated to the patient age in terms of the composition of collagens in the decellularized tumor tissue (FIG. 7).

In a particular embodiment, analyzing protein composition comprises analyzing protein composition of decellularized tumor tissues obtained from multiple tumors characterized by the at least one defined tumor property or from tumors of multiple patients characterized by the at least one defined patient property.

Hence, in this embodiment protein composition is analyzed in a set of multiple, i.e., at least two, decellularized tumor tissues. More preferably, the set comprises at least 3, at least 5 or more, such as at least 6, 7, 8, 9 or 10, or even more such as at least 15 or 20 decellularized tumor tissues obtained from different tumors. The higher the number of decellularized tumor tissues analyzed in the method the more accurate statistical relevance of the proteins selected to be differentially expressed in the decellularized tumor tissue as compared to the reference tissue or tissues.

The analysis of protein composition of the decellularized tumor tissue can be performed according to various technologies for protein composition analysis. Non-limiting, but illustrative, examples of such technologies include mass spectrometry (MS) and various immunoassays, such as enzyme-linked immunosorbent assay (ELISA), immunostaining, dot blot methods, Western blot, proximity extension assay (PEA), and other immunohistochemistry (IHC) and immunocytochemistry (ICC) methods.

The method selects at least one protein, i.e., one or multiple, i.e., at least two, proteins that is differently expressed in the decellularized tumor tissue or tissues as compared to a reference tissue or tissues. Differently expressed as used herein means that the protein or proteins is present in a different amount or at a different level or concentration in the decellularized tumor tissue(s) as compared to the reference tissue(s). A differently expressed protein can be higher expressed in the decellularized tumor tissue(s) as compared to the reference tissue(s) and is thereby present in a higher amount, level or concentration in the decellularized tumor tissue(s) as compared to the reference tissue(s). Alternatively, a differently expressed protein can be lower expressed in the decellularized tumor tissue(s) as compared to the reference tissue(s) and is thereby present in a lower amount, level or concentration in the decellularized tumor tissue(s) as compared to the reference tissue(s).

In an embodiment, one protein is selected. In another embodiment, more than one protein is selected, such as 2, 3, 4, 5, 6, 7, 8, 9, 10 proteins. It is typically sufficient to select from 1 up to 10 proteins that are differently expressed in the decellularized tumor tissue or tissues as compared to a reference tissue or tissues. However, the embodiments are not limited thereto. For instance, up 15 proteins, up to 20 proteins, up to 25 proteins or even up to 50 proteins could be selected.

The protein amount, level or concentration in the decellularized tumor tissue(s) is determined based on the analysis of the protein composition of the decellularized tumor tissue(s), such as using any of the above exemplified technologies.

The corresponding protein amount, level or concentration in the reference tissue(s) may, in an embodiment, be retrieved from a table or other information source listing protein amounts, levels or concentrations in such reference tissues. This information of protein amounts, levels or concentrations may, for instance, have been previously determined in the reference tissue(s) using any of the above exemplified technologies. The reference tissue(s) could then be a non-decellularized tissue sample or samples, but is preferably a decellularized tissue sample or samples.

In another embodiment, the method comprises analyzing protein composition of a reference decellularized tissue or tissues obtained from the reference tissue or tissues. The analysis of protein composition of the reference decellularized tissue(s) is preferably performed in the same or at least similar way as the analysis of the protein composition of the decellularized tumor tissue(s).

In an embodiment, the reference tissue(s) is a non-tumor tissue or tissues. An illustrative, and preferred, example of such non-tumor tissue is a non-tumor tissue present adjacent to the tumor in a patient from which the tumor is obtained. Hence, when retrieving the tumor tissue sample from a patient, the sample may contain both the actual tumor tissue but also non-tumor tissue, i.e., healthy tissue, present adjacent to or in the vicinity of the tumor in the patient. The so obtained sample then comprises both tumor tissue and the non-tumor, reference tissue.

Decellularization of the sample(s) retrieved from the patient(s) then results in decellularized tumor tissue(s) and adjacent decellularized non-tumor tissue(s), i.e., reference decellularized tissue(s). In such a case, analysis of the protein composition can be performed on the same sample(s) to determine the protein composition of the decellularized tumor tissue(s) in the sample and determine the protein composition of the reference decellularized non-tumor tissue(s).

In another embodiment, the reference tissue(s) is at least one reference tumor not characterized by the at least one defined tumor property or is from at least one patient not characterized by the at least one defined patient property. In this embodiment, both the tumor tissue(s) and the reference tissue(s) are obtained from tumors but from tumors having different tumor properties or from tumors in patients having different patient properties, such as age. An illustrative, but non-limiting, example of the former is tumor tissue obtained from a high grade tumor versus reference tissue obtained from a low grade tumor. An illustrative, but non-limiting, example of the latter is tumor tissue obtained from an old patient versus reference tissue obtained from a tumor in a comparatively younger patient.

In a particular embodiment, reference tissue(s) is decellularized tumor tissue(s) obtained from at least one reference tumor not characterized by the at least one defined tumor property or obtained from at least one patient not characterized by the at least one defined patient property.

The discussion above in relation to analyzing protein composition in a set of decellularized tumor tissues can also be applied to analyzing protein composition in a set of reference tissues, such as a set of reference decellularized tissues.

Illustrative, but non-limiting, examples of tumors from which the decellularized tumor tissue is obtained and reference tumors from which the reference (decellularized) tissue is obtained include the tumor is a grade III tumor and the reference tissue is a grade I or II tumor; the tumor is one of an invasive ductal carcinoma (IDC) and invasive lobular carcinoma (ILC) and the reference tissue is the other of IDS and ILC; the tumor is one of an ERα-negative tumor and an ERα-positive tumor and the reference tissue is the other of ERα-negative tumor and ERα-positive tumor; the tumor is one of an PR-negative tumor and a PR-positive tumor and the reference tissue is the other of PRα-negative tumor and PR-positive tumor; the tumor is a tumor having a size or volume larger than a threshold size or volume and the reference tissue is a tumor having a size or volume smaller than the threshold size/volume; and the tumor is a malignant tumor and the reference tissue is a non-malignant tumor. In these illustrative examples, the reference tumor(s) is (are) thereby used for comparative analysis in terms of determining the differences between tumors having different tumor characteristics or properties.

In an embodiment, the method also comprises decellularizing tumor tissue(s) obtained from at least one tumor to form or obtain the decellularized tumor tissue(s). Decellularizing methods often employ a prolonged mild detergent treatment of the tumor tissue as disclosed in Example 1. Merely by way of example, a decellularizing method may comprise subjecting a suitable tumor sample, such as a section taken from a tumor sample, to one or more, for instance two, three, four or more, detergent or lysis washes, often referred to as decellularization cycles. Any suitable detergent may be used in the detergent or lysis buffer including, but not limited to SDS, Triton X-100, NP40, and/or TWEEN® 20. Optionally, after each cycle, a small tissue extract may be screened for the presence or absence of cells, for example, by screening for nuclei. Typically, the decellularization cycles are continued until cells are not detectable.

Decellularization cycles may be followed by one or more washes to remove cell debris using, for instance, distilled water, a buffer solution or the detergent or lysis buffer but excluding the detergent. A decellularized tumor tissue may be sterilized using a suitable sterilizing agent, including, but not-limited to, peracetic acid, antibiotics, etc.

The above described decellularizing method may also be applied to the reference tissue(s) to obtain the reference decellularized tissue(s).

In an embodiment, tumor tissue is obtained from a solid tumor and is decellularized to form the decellularized tumor tissue. In a particular embodiment, the solid tumor is selected from the group consisting of a breast cancer tumor, a colon cancer tumor, an ovarian cancer tumor, a lung cancer tumor, and a pancreatic cancer tumor.

In an embodiment, producing the bioink comprises producing the bioink comprising the selected at least one protein, or a domain thereof, and at least one ECM protein, or a structural domain thereof. Hence, in this embodiment, the at least one protein selected as being differently expressed in the decellularized tumor tissue as compared to the reference tissue is at least one non-ECM protein. The bioink then comprises the selected at least one non-ECM protein, or a domain thereof, and at least one ECM protein, or a structural domain thereof.

In such an embodiment, the at least one ECM protein provides structural functional to the 3D scaffold as produced from the bioink whereas the at least one selected (non-ECM) protein implies that the 3D scaffold mimics the defined tumor property of a tumor and/or the patient property, such as age of the patient.

The at least one ECM protein, or the structural domain thereof, is preferably included to render the 3D scaffold rigid enough for allowing three-dimensional culturing of cells in the 3D scaffold. The amount of and/or types of such ECM proteins can be selected to tailor the rigidity of the produced 3D scaffold.

In an embodiment, the bioink comprises a single ECM protein, or a structural domain thereof. In another embodiment, the bioink comprises multiple different ECM proteins, or structural domains thereof.

Illustrative, but non-limiting, examples of ECM proteins include collagen, elastin, fibronectin, and laminin.

In an embodiment, the bioink comprises at least one keratin, or a structural domain thereof, instead of or as a complement to the at least one ECM protein, or the structural domain thereof.

In an embodiment, producing the bioink comprises producing the bioink comprising the selected at least one protein, or a domain thereof, and at least one component selected from the group consisting of chitosan, alginate, fibrinogen, cellulose, and any combination thereof. This embodiment may also be combined with the embodiment above so that the bioink comprises the selected at least one (non-ECM) protein, or a domain thereof, at least one ECM protein, or a structural domain thereof, and at least one component selected from the group consisting of chitosan, alginate, fibrinogen, cellulose, and any combination thereof.

Bioink base component as used herein provides the structural integrity and form the physical structure of the produced 3D scaffold. For instance, the bioink base component may form fibers when producing the 3D scaffold from the bioink. In a particular embodiment, the bioink base component is selected from, but not limited to, the group consisting of an ECM protein, or a structural domain thereof, at least one component selected from the group consisting of chitosan, alginate, fibrinogen, cellulose, and any combination thereof.

In an embodiment, at least one bioink base component is selected based on a production method used to produce the 3D scaffold using the bioink. In such an embodiment, the bioink is produced to comprise the selected at least one protein, or the domain thereof, and the selected at least one bioink base component. Furthermore, the bioink is produced using the production method to form the 3D scaffold mimicking a tumor characterized by the at least one defined tumor property or a tumor a patient characterized by the at least one defined patient property.

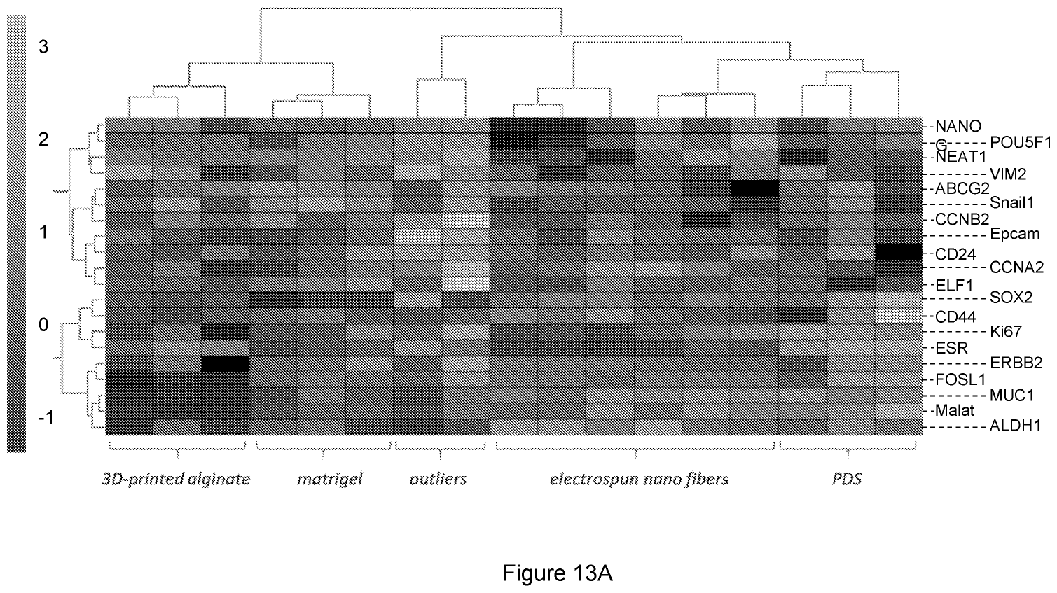

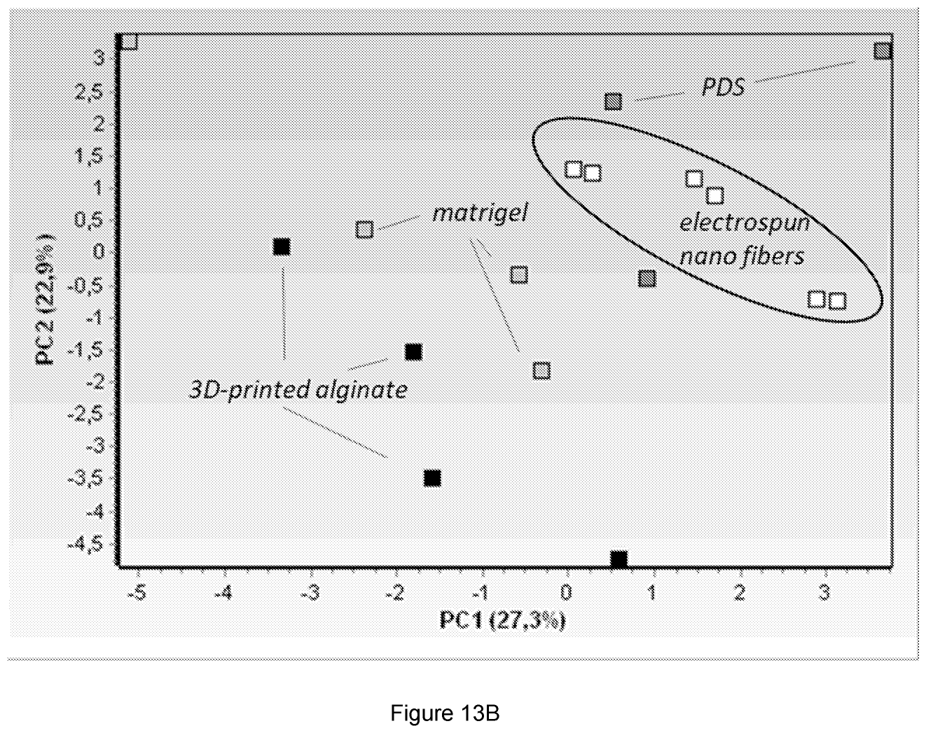

Experimental data as presented herein (FIG. 13) indicate that the particular production or manufacturing method used to produce the 3D scaffold from the bioink affects gene expression of cancer cells cultured in the 3D scaffolds. For instance, the gene expression profile of cancer cells cultured in electrospun 3D scaffold was more similar to the gene expression of cancer cells cultured in a patient-derived decellularized tumor tissue as compared to the other tested production methods.

The composition of the bioink base components is preferably selected based on the particular production method used to produce the 3D scaffold from the bioink. This selection is thereby tailors the particular base components for the bioink and thereby for the 3D scaffold to be adapted to the production method and thereby result in a 3D scaffold that, together with the at least one selected protein, or the domain thereof, mimics a tumor a tumor characterized by the at least one defined tumor property or a tumor a patient characterized by the at least one defined patient property.

In an embodiment, the production method is selected from the group consisting of electrospinning, wet spinning, melt spinning, 3D printing, pouring, phase separation, particle leaching, and any combination thereof. In a particular embodiment, the production method is an additive manufacturing (AM) method or process, sometimes referred to as additive layer manufacturing (ALM) or 3D printing. Such AM or ALM methods adds material to create an object, in this case adds bioink to create the 3D scaffold. AM or ALM method often uses computer-aided-design (CAD) software or 3D scanners to direct hardware to deposit material, in this case bioink, layer upon layer, in precise geometric shapes. In an embodiment, the AM or ALM method is selected from the group consisting of vat photopolymerization, material jetting, binder jetting, material extrusion, powder bed fusion, sheet lamination and direct energy deposition.

In a particular embodiment, the production method is a combination of electrospinning and 3D printing. Such a combination of 3D printing and electrospinning could combine the characteristics of both production processes. For instance, electrospinning enables production of micro-to nanometer fibers, of which the dimensions, alignment, porosity and chemical composition are manipulatable to the desired application. 3D printing then enables production of 3D scaffolds having a comparatively complex physical structure. For instance, a multi-layer 3D scaffold could be produced by inclusion of electrospun fibers in bioinks used for 3D printing or by alternate between producing layers by electrospinning and 3D printing, which is further described herein.

The protein composition of decellularized tumor tissues obtained from tumors of different cancer types, such as breast cancer (breast cancer tumors), colon cancer (colon cancer tumors), ovarian cancer (ovarian cancer tumors), pancreatic cancer (pancreatic cancer tumors), etc., may at least partly overlap. Hence, a set of proteins may be differently expressed in the decellularized tumor tissues obtained from tumors of all of these cancer types as compared to reference tissues, whereas other proteins may be specific for decellularized tumor tissue obtained from tumors of a specific type. As an example, decellularized tumor tissues obtained from, for instance, breast and colon cancer tumors may have a first set of common proteins that is differentially expressed as compared to reference tissue(s). Furthermore, a second set of proteins is differently expressed in decellularized tumor tissue obtained from breast cancer tumors as compared to reference tissue(s) but not differently expressed in decellularized tumor tissue obtained from colon cancer tumors as compared to reference tissue(s). Correspondingly, a third set of proteins is differently expressed in decellularized tumor tissue obtained from colon cancer tumors as compared to reference tissue(s) but not differently expressed in decellularized tumor tissue obtained from breast cancer tumors as compared to reference tissue(s).

In such a case, the protein(s) differentially expressed in decellularized tumor tissues obtained from tumors of different cancer types could then be included in a pan-cancer bioink for the production of a pan-cancer 3D scaffold. One or more proteins that are differently expressed only in decellularized tumor tissue obtained from tumors of a specific cancer type could then be included in such a pan-cancer bioink to get a cancer type-specific bioink for the production of a cancer-specific 3D scaffold.

The bioink of the embodiments comprising the selected at least one protein can be used to produce a 3D scaffold mimicking a tumor characterized by the at last one defined tumor property or a tumor of a patient characterized by the at least one defined patient property. Hence, it is possible to obtain such a 3D scaffold mimicking a real tumor and tumor microenvironment using merely a single selected protein or a limited number of selected proteins. Hence, by selecting one or multiple proteins that are differentially expressed in the decellularized tumor tissue(s) as compared to the reference tissue(s) it is possible to obtain or achieve the desired characteristics or properties of the real tumor or tumor microenvironment, such as represented by a patient-derived decellularized tumor samples (PDSs). However, as compared to using PDSs derived from tumors, the 3D scaffolds of the invention can be produced in a reproducible way by having a well-defined composition of the bioink. In this way, by producing bioinks for certain purposes, e.g., tumor properties, the response of the cancer cells to pharmacological intervention based on the microenvironment provided by the 3D scaffold can be directly compared between tumors of different ‘types’ or properties, e.g., between aggressive tumors and non-aggressive tumors, or between cancer types, e.g., breast cancer and colon cancers, or between recurring and non-recurring tumors, or how different medicaments will affect tumor tissue representing different patient age.

Experimental data as presented herein indicates that there is a correlation between ECM proteins in a decellularized tumor tissue and tumor and histological grade of the tumor from which the decellularized tumor tissue is obtained. Hence, the proportion and/or mixture of such ECM proteins in a bioink could be used to tailor the predicted tumor or histological grade of the 3D scaffold produced from the bioink.

An aspect of the invention therefore relates to a method for producing a bioink for production of a 3D scaffold. The method comprises selecting a predicted tumor or histological grade for the 3D scaffold. The method also comprises determining a proportion of proteins in the 3D scaffold that are ECM proteins and/or a mixture of ECM proteins based on the selected tumor grade. The method further comprises producing a bioink comprising the determined proportion and/or mixture of ECM proteins, or structural domains thereof. The 3D scaffold produced from the produced bioink mimics a tumor of the selected tumor or histological grade.

In an embodiment, determining the proportion and/or mixture comprises determining a first proportion of ECM proteins for a selected first tumor or histological grade and determining a second, higher proportion of ECM proteins for a selected second, lower tumor or histological grade.

Hence, in this embodiment, decellularized tumor tissue obtained from low grade tumors generally had a higher proportion of ECM proteins as compared to analyzed decellularized tumor tissue from high grade tumors. This means that a 3D scaffold obtained from a bioink having a low proportion of ECM proteins, or structural domains thereof, mimics properties of high grade tumors, whereas a 3D scaffold obtained from a bioink having a comparatively higher proportion of ECM proteins, or structural domains thereof, mimics properties of low grade tumors.

The proportion of ECM proteins in a bioink or 3D scaffold as used herein corresponds, in an embodiment, to the number of different ECM proteins in a bioink or 3D scaffold divided by the total number of different proteins in the bioink or 3D scaffold. For instance, a bioink or 3D scaffold comprising three different ECM proteins and 12 different non-ECM proteins has a proportion of ECM proteins of 3/(3+12)=0.2 or 20%. The proportion of ECM proteins in a bioink may alternatively be weight of different ECM proteins in a bioink or 3D scaffold divided by the total weight of different proteins in the bioink or 3D scaffold, e.g., w/w %, or indeed weight of different ECM proteins in a bioink divided by the volume of the bionk, e.g., w/v %.

In an embodiment, determining the proportion and/or mixture comprises determining a first mixture of ECM proteins for a selected first tumor grade and determining a second, different mixture of ECM proteins for a selected second, lower tumor grade.

Experimental data as presented herein indicates that there is a correlation between the amount, and/or type, of keratins in a decellularized tumor tissue and recurrence status of the tumor from which the decellularized tumor tissue is obtained. Hence, the amount, and/or type, of keratins in a bioink could be used to tailor the recurrence status of the 3D scaffold produced from the bioink.

An aspect of the invention therefore relates to a method for producing a bioink for production of a 3D scaffold. The method comprises (a) determining whether the 3D scaffold is representative of a recurring tumor or a non-recurring tumor. The method also comprises (b) determining an amount, and/or type, of keratins in the 3D scaffold based on the determination in (a). The method further comprises (c) producing a bioink comprising the determined amount, and/or type, of keratins, or a structural domain thereof. The 3D scaffold produced from the produced bioink mimics a recurring tumor or a non-recurring tumor.

In an embodiment, determining the amount, and/or type, of keratins comprises determining a first amount of keratins if the 3D scaffold is determined to be representative of a recurring tumor and determining a second, lower amount of keratins if the 3D scaffold is determined to be representative of a non-recurring tumor.

Hence, in this embodiment, decellularized tumor tissue obtained from recurring tumors generally had a higher amount of keratins as compared to analyzed decellularized tumor tissue from non-recurring tumors. This means that a 3D scaffold obtained from a bioink having a higher amount of keratins, or structural domains thereof, mimics properties of recurring tumors, whereas a 3D scaffold obtained from a bioink having a comparatively lower amount of keratins, or structural domains thereof, mimics properties of non-recurring tumors.

In an embodiment, the keratins are selected from the group consisting of keratin 1, keratin 2, keratin 9, keratin 10, keratin 14, keratin 16 and keratin 78. Hence, decellularized tumor tissue from recurring tumors had significantly higher amounts of keratin 1, keratin 2, keratin 9, keratin 10, keratin 14, keratin 16 and keratin 78 as compared to decellularized tumor tissue from non-recurring tumors.

In an embodiment, determining the amount, and/or type, of keratins comprises determining a first type of keratins if the 3D scaffold is determined to be representative of a recurring tumor, such as selecting one or more keratins from the above presented group, and determining a second different type of keratins if the 3D scaffold is determined to be representative of a non-recurring tumor, such as selecting one or more keratins different from the keratins listed in the above presented group.

In an embodiment, determining the amount of keratins in the 3D scaffold based on the determination in (a) comprises determining the amount of one keratin in the above presented group, determining the amount of two keratins in the above presented group or determining the amount of three or more of the keratins in the above presented group, such as determining the amount of four, five, six or all seven keratins in the above presented group.

Experimental data as presented herein indicates that there is a correlation between the amount of lacritin in a decellularized tumor tissue and recurrence status of the tumor from which the decellularized tumor tissue is obtained. Hence, the amount of lacritin in a bioink could be used to tailor the recurrence status of the 3D scaffold produced from the bioink.

An aspect of the invention therefore relates to a method for producing a bioink for production of a 3D scaffold. The method comprises (a) determining whether the 3D scaffold is representative of a recurring tumor or a non-recurring tumor. The method also comprises (b) determining an amount of lacritin in the 3D scaffold based on the determination in (a). The method further comprises (c) producing a bioink comprising the determined amount of lacritin, or a domain thereof. The 3D scaffold produced from the produced bioink mimics a recurring tumor or a non-recurring tumor.

In an embodiment, determining the amount of lacritin comprises determining a first amount of lacritin if the 3D scaffold is determined to be representative of a non-recurring tumor and determining a second, higher amount of lacritin if the 3D scaffold is determined to be representative of a recurring tumor.

Hence, in this embodiment, decellularized tumor tissue obtained from non-recurring tumors from patients having a comparatively longer disease-free survival generally had a lower amount of lacritin as compared to analyzed decellularized tumor tissue from recurring tumors from patients with a comparatively shorter disease free survival. This means that a 3D scaffold obtained from a bioink having a lower amount of lacritin, or a domain thereof, mimics properties of non-recurring tumors from patients with longer disease-free survival, whereas a 3D scaffold obtained from a bioink having a comparatively higher amount of lacritin, or a domain thereof, mimics properties of recurring tumors from patients with shorter disease-free survival.

Lacritin may be present in different lacritin splice variants. In an embodiment, step (b) in the above-described method may, thus, comprise determining the type of lacritin in the 3D scaffold, instead of or as a complement to determining the amount of lacritin, based on the determination in (a). Step (c) then comprises producing a bioink comprising the determined amount, and/or type of lacritin, or a domain thereof.

The various embodiments of the invention as described above may be combined. Hence, bioinks can be produced to comprise a proportion and/or mixture of ECM proteins, or structural domains thereof, determined based on a selected tumor or histological grade and/or an amount, and/or type, of keratins, or structural domains thereof, determined based on whether the 3D scaffold should mimic a recurring or non-recurring tumor and/or an amount of lacritin, or domains thereof, determined based on whether the 3D scaffold should mimic a recurring or non-recurring tumor and/or proteins, or domains thereof, that are differently expressed in decellularized tumor tissue(s) as compared to reference tissue(s).

For instance, the method for producing a bioink comprising a determined amount, and/or type, of keratins, or a structural domain thereof, and/or a determined amount of lacritin, or a domain thereof, may optionally comprise an additional step of adding ECM proteins, or a structural domain thereof, to obtain a proportion of ECM proteins, or a structural domain thereof, in the bioink selected based on a selected or defined tumor or histological grade of the tumor. In another embodiment, the method may optionally comprise an additional step of adding a mixture of ECM proteins, or structural domains thereof, to the bioink selected based on a selected or defined tumor or histological grade of the tumor.

The present invention also relates to a method for producing a 3D scaffold. The method comprises producing a bioink according to any of the embodiments disclosed herein and producing the 3D scaffold using the bioink.

In an embodiment, the 3D scaffold is produced using the bioink using any of the above-mentioned production methods including, but not limited to, electrospinning, wet spinning, melt spinning, 3D printing, pouring, phase separation, particle leaching, and any combination thereof. In a particular embodiment, the 3D scaffold is produced using electrospinning, 3D printing (AM or ALM), or a combination thereof.

Hence, in an embodiment, the method comprises bioprinting the bioink by a 3D printer to produce the 3D scaffold.

Bioprinting a 3D scaffold by a bioink of the present invention can be performed using any commercially available bioprinter apparatus, such as the 3D bioprinter INKREDIBLE or INKREDIBLE+ by Cellink AB.

Typically, bioprinting the 3D scaffold is performed under physiological conditions. More specifically, the temperature and printing pressure during bioprinting could be selected within an interval of from 4° C. to 40° C. and from 1 kPa to 200 kPa, respectively.

Also, a cross-linking reagent may be used during or after the bioprinting process. A non-limiting, but illustrative, example of such a cross-linking reagent could be CaCl₂), such as in the form of a 100 mM CaCl₂) solution.

In another embodiment, the method comprises electrospinning the bioink to produce the 3D scaffold.

In a particular embodiment, the method comprises electrospinning a bioink base component to form fibers, in particular micro- and/or nanofibers. The method also comprises adding the electrospun fibers to the bioink and bioprinting the bioink comprising the electrospun fibers by a 3D printer to produce the 3D scaffold. For instance, electrospinning can be used to produce ECM-mimicking fibers that are included in the bioink.

In another particular embodiment, the method comprises electrospinning a bioink according to the invention to form fibers, in particular micro- and/or nanofibers. The method also comprises adding the electrospun fibers to the bioink and bioprinting the bioink comprising the electrospun fibers by a 3D printer to produce the 3D scaffold. Hence, in this particular embodiment, the electrospun fibers are made of the same bioink as used during bioprinting.

In a further particular embodiment, the method comprises electrospinning a first bioink to form fibers, in particular micro- and/or nanofibers. The method also comprises adding the electrospun fibers to a second bioink and bioprinting the second bioink comprising the electrospun fibers by a 3D printer to produce the 3D scaffold. Hence, in this particular embodiment, the electrospun fibers are made of a different bioink that what is used during bioprinting.

In yet another embodiment, the method comprises electrospinning or bioprinting a bioink according to the invention to form a first layer of the 3D scaffold followed by bioprinting or electrospinning the bioink, or another bioink according to the invention, to form a second layer on the first layer. This process can then proceed multiple times by alternating between electrospinning and bioprinting to produce the 3D scaffold layer by layer. The method does not necessarily need to alternate between electrospinning and bioprinting for each layer. For instance, multiple layers of the 3D scaffold can be produced one of electrospinning and bioprinting followed by producing one or multiple layers using the other of electrospinning and bioprinting and then optionally once more producing one or multiple layers using the one of electrospinning and bioprinting.

In a further embodiment, the method comprises electrospinning or bioprinting a bioink according to the invention to form a layer of fibers for the 3D scaffold followed by bioprinting or electrospinning the bioink, or another bioink according to the invention, to mesh network interconnecting the fibers in the layer. Another layer of fibers can then be electrospun or bioprinted on the layer of fibers and the fibers in this another layer of fibers are interconnected by a bioprinted or electrspun mesh network. As an example, bioprinting can produce a layer of microfibers, which are interconnected by a mesh network of electrospun nanofibers.

Microfiber as used herein relates to a fiber having an average diameter in the micrometer range, typically an average diameter equal to or less than 100 μm, preferably equal to or less than 10 μm. Correspondingly, nanofiber relates to a fiber having an average diameter in the nanometer range.

In an embodiment, the method comprises selecting at least one tumor property of the 3D scaffold. The method then comprises selecting a production method for producing the 3D scaffold using the bioink with the selected production method.

In an embodiment, the at least one scaffold property is selected from the group consisting of average fiber diameter of fibers comprised in the 3D scaffold, fiber arrangement, average pore size and pore structures or pore distribution in the 3D scaffold.

The size of the produced 3D scaffold can be selected based on the particular use and application of the 3D scaffold in cancer research. Generally, the 3D scaffold may have a diameter, length and/or width selected within an interval of from 100 μm up to 10 cm, typically from 500 μm up to 50 mm, such as from 0.75 mm up to 50 mm. As an illustrative example, the 3D scaffold could have a diameter or width of 15 to 20 mm, or less, and a thickness or height of 1 to 5 mm.

In an embodiment, the bioink can be based on any natural and/or synthetic polymer(s) selected for its/their biocompatible components and favorable rheological properties. These characteristics temporarily or permanently support living cells to facilitate their adhesion, proliferation and differentiation during maturation. A bioink should provide binding sites for cells, while promoting their own ECM production and ultimately help in generation of a functional tissue. Besides excellent bioactive properties, the bioink should be, for instance, printable or spinnable to ensure that the bioink can be extruded through a printing nozzle or needle into filaments while maintaining the shape fidelity and hierarchical deposit of the resulting 3D scaffold.

The composition of the bioink for producing reproducible 3D scaffolds for use as in vitro models in cancer research applications is selected to mimic desired tumor characteristics for the 3D scaffold. The bioink, thus, comprises the selected at least one protein, or a domain thereof, which is differentially expressed, as compared to a reference tissue or tissues, in decellularized tumor tissue(s) obtained from at least one tumor characterized by at least one defined tumor property or from a tumor of a patient characterized by at least one defined patient property.

After selecting the components of the bioink, the relative amounts of the components and the cross-linking conditions are selected or systematically optimized in repeated production experiments to obtain a 3D scaffold suitable for culturing of cells, in particular cancer cells. A proportion of the components of the bioink is selected for structural support. Such structural support components are selected based on biocompatibility, cost and facile gelation. One such component may be alginate, which is widely used for 3D scaffolds, but other structural components as described herein can be added or used instead of alginate, including, but not-limited to, cellulose, and MATRIGEL®. The structural components, including any combination of ECM proteins selected for the desired 3D scaffold characteristics, should be sufficient to allow production of the 3D scaffold and to maintain the 3D shape fidelity during the production process. The bioink is optionally also optimized to enable extrusion printing at reasonable pressure and to preserve 3D structural integrity during the procedure.

The properties of bioinks may be improved by blending different biopolymers with distinct characteristics. Such mixtures can be used to combine the printability and structural stiffness with high cell viability and metabolic activity of the (cancer) cells to be cultured in the 3D scaffold. Frequently, blends of alginate and gelatin are used for extrusion-based bioprinting to combine the thermo-sensitive properties of gelatin with the chemical cross-linking capabilities of alginate.

Bioprinting 3D scaffolds using the bioink comprising selected proteins is then performed according to techniques well known in the art, such as disclosed in Svanström et al., Optimized alginate-based 3D printed scaffolds as a model of patient derived breast cancer microenvironments in drug discovery, Biomedical Materials, 24 May 2021 (doi: 10.1088/1748-605X/ac0451), the teaching of which regarding 3D-Printing on pages 2-3 is hereby incorporated by reference.

The present invention further relates to a bioink obtainable by any of the methods as disclosed herein.

Another aspect of the invention relates to a bioink comprising at least one protein, or a domain thereof, which is differentially expressed in a decellularized tumor tissue or tissues as compared to a reference tissue or tissues.

In an embodiment, the reference tissue or tissues is or are selected from a non-tumor tissue or tissues and a reference tumor or tumors i) having at least one tumor property different than at least one tumor from which the decellularized tumor tissue or tissues is or are obtained or ii) from a respective patient having at least one patient property different than a respective patient from which at least one tumor from which the decellularized tumor tissue or tissues is or are obtained.

In an embodiment, the bioink additionally comprises at least one ECM protein, or a structural domain thereof.

In a particular embodiment, a quantity of the ECM protein, or the structural domain thereof, in the bioink is selected based on a desired tumor grade of the 3D scaffold.

In an embodiment, the bioink comprises one or more of the following protein components:
at least one collagen, or a structural domain thereof, preferably selected from the group consisting of collagen alpha-1 (I) chain, collagen alpha-1 (III) chain, collagen alpha-1 (V) chain, collagen alpha-1 (XVI) chain, collagen alpha-2 (I) chain, collagen alpha-2 (V) chain, or a structural domain thereof;
elastin, or a structural domain thereof;
at least one laminin, or a structural domain thereof, preferably selected from the group consisting of laminin subunit alpha, laminin subunit beta, or a structural domain thereof;
at least one latent-transforming growth factor beta-binding protein, or a structural domain thereof, preferably latent-TGFβ-binding protein 4, or a structural domain thereof;
versican, or a structural domain thereof;
dermatopontin, or a structural domain thereof; and
at least one elastin microfibril interface, or a structural domain thereof, preferably EMILIN-1, or a structural domain thereof.

The above-described additional protein components of the bioink are common protein components in decellularized tumor tissue obtained from tumors of different types and having different tumor properties. Hence, these additional protein components could be regarded as basic protein components (bioink base components) of decellularized tumor tissue and thereby suitable as basic protein components for a bioink and a 3D scaffold produced from such a bioink.

These proteins are all ECM proteins or are at least forming a part of the ECM of tumor tissue.

In another embodiment, the bioink lacks any ECM proteins, or any structural domain thereof.

In an embodiment, the bioink further comprises a chymase and/or a peroxiredoxin, such as peroxiredoxin-4, or a functional domain thereof.

Chymases (EC 3.4.21.39) are a family of serine proteases found primarily in mast cells, though also present in basophil granulocytes. They show broad peptidolytic activity and are involved in a variety of functions including inflammation. Peroxiredoxin (EC 1.11.1.15) are a ubiquitous family of antioxidant enzymes that also control cytokine-induced peroxide levels and thereby mediate signal transduction in mammalian cells. Peroxiredoxins reduce hydrogen peroxide and alkyl hydroperoxides to water and alcohol with the use of reducing equivalents derived from thiol-containing donor molecules. Peroxiredoxin-4 has been found to play a regulatory role in the activation of the transcription factor NF-κB.

Also chymases and peroxiredoxins are common protein components in many decellularized tumor tissues and may therefore advantageously be included in the bioink.

The present invention is also directed towards a bioink comprising proteins, or domains thereof, wherein a proportion of the proteins in the bioink that are ECM proteins, or structural domains thereof, is selected based on a defined tumor or histological grade. In this embodiment, a 3D scaffold produced by the bioink then mimics a tumor of the defined tumor or histological grade.

In a particular embodiment, the proportion of ECM proteins in the bioink is less than 15%, preferably less than 10%, and more preferably less than 8%, and even more preferably less than 5%. The percentage may be w/w % or w/v %.

An embodiment of the invention relates to a bioink comprising lacritin, or a domain thereof.

In a particular embodiment, the amount, of lacritin in the bioink is selected based on a whether a 3D scaffold produced by the bioink should mimic a recurring tumor or a non-recurring tumor.

Another embodiment of the invention relates to a bioink comprising keratins, or a structural domain thereof.

In a particular embodiment, the amount, and/or type, of keratins in the bioink is selected based on a whether a 3D scaffold produced by the bioink should mimics a recurring tumor or a non-recurring tumor.

The bioink according to any of the above-described embodiments may optionally comprise at least one component selected from the group consisting of chitosan, alginate, fibrinogen, cellulose, and any combination thereof.

The invention also relates to a 3D scaffold for cell culturing obtainable from the bioink according to any of the embodiments.

In an embodiment, the 3D scaffold is obtainable by bioprinting the bioink according to any of the embodiments by a 3D printer. In another embodiment, the 3D scaffold is obtainable by electrospinning the bioink according to any of the embodiments. In a further embodiment, the 3D scaffold is obtainable by bioprinting and electrospinning.

In an embodiment, the 3D scaffold mimics a tumor characterized by the at least one defined tumor property or a tumor of a patient characterized by at least one defined patient property.

In a particular embodiment, the at least one defined tumor property is selected from the group consisting of tumor grade, tumor size, cancer type or subtype, ER status, PR status, and recurrence, preferably selected from the group consisting of tumor grade, tumor size, ER status, PR status, and recurrence.

In another particular embodiment, the at least one defined patient property is the age of the patient.

A further aspect of the invention is a cell culturing method. The method comprises adding cells to a 3D scaffold according to above and culturing the cells in the 3D scaffold.

The cell culturing method is an in vitro method. Hence, cells are added in vitro to the 3D scaffold and cultured in vitro in the 3D scaffold.

Culturing of cells in the 3D scaffold are preferably conducted in an environment and conditions generally applicable for in vitro cell culturing, such as in a temperature of about 37° C. in 5% CO₂.

In an embodiment, the cells added to and cultured in the 3D scaffold are cancer cells. In another embodiment, the cells added to and cultured in the 3D scaffold are non-cancerous cells, such as leukocytes or patient-derived non-cancerous cells. In a further embodiment, a combination of cells, such as cancer cells and non-cancerous cells, such as leukocytes and/or patient-derived non-cancerous cells, is added to and cultured in the 3D scaffold.

The microenvironment of the 3D scaffold is not just a physical scaffold providing three-dimensional structure for cell culturing, but it is produced by the selected components of the bioink to affect, i.e., modulate, the way that the cultured cells behave in a predicted manner, i.e., their gene expression, including their expression of certain biomarkers when these cells are cultured in the 3D scaffold. Furthermore, this modulation of the gene expression, including the expression of biomarkers, provides valuable information of the tumor tissue, which the 3D scaffold mimics. For instance, there is a correlation between expression of various biomarkers by cancer cells and tumor properties relevant for the tumor and the cancer disease. This means that the manufactured 3D scaffold can be used as an in vitro model of the specific microenvironment of a specific type of cancer and/or a specific type of tumor and it can be used as a tool to determine such tumor properties by analyzing the expression of at least one biomarker in the cancer cells when being, or having been, cultured in the 3D scaffold.

The analysis of the expression of the at least one biomarker can be performed according to various embodiments. Illustrative examples include analysis on the RNA level, such as using a gene expression array that measures the levels of gene products, i.e., mRNA transcripts, from selected genes encoding biomarkers. Such gene expression arrays are also denoted DNA (micro) array in the art. Alternatively, gene expression can be analyzed using quantitative polymerase chain reaction (qPCR) or by gene sequencing. A further alternative is to use an immunoassay, i.e., detecting and preferably quantifying, immunomarkers using antibodies that specifically bind to the respective immunomarkers. Such immunoassays include ELISA, immunostaining, Luminex immunoassay, dot blot methods, Western blot and other IHC and ICC methods. Further techniques include mass spectrometry, fluorescence-activated cell sorting (FACS), magnetic-activated cell sorting (MACS), proximity extension assay (PEA), and proximity ligation assay (PLA).

Cellular property or characteristic as used herein relates to a property or characteristic of the cells, such as cancer cells, cultured in the 3D scaffold. Illustrative examples of such cellular property or characteristic include cell morphology, cell size, cellular motility, cellular invasiveness, etc.

In an embodiment, the method comprises exposing the cancer cells to a cancer treatment while culturing the cancer cells in the 3D scaffold. The method also comprises, in this embodiment, determining a response of the cancer cells to the cancer treatment.

In this particular embodiment, the cancer cells cultured in the 3D scaffold are exposed to a cancer treatment. The cancer treatment applied to the cancer cells cultured in the 3D scaffold can be any cancer treatment that mimics a cancer treatment that can be applied in vivo to the subject. For instance, the cancer treatment could be selected from the group consisting of chemotherapy, radiation therapy, immunotherapy, targeted cancer therapy, and any combination thereof.

Chemotherapy is a type of cancer treatment that uses one or more anti-cancer drugs, i.e., chemotherapeutic agents, including, but not limited to, alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursors thereof, peptide antibiotics, platinum-based agents, retinoids and vinca alkaloids and derivatives thereof. In a particular embodiment, exposing the cancer cells to a cancer treatment comprises adding at least one chemotherapeutic agent to the culture medium, in which the cancer cells are cultured in the 3D scaffold.

Radiation therapy, also referred to as radiotherapy, is a cancer treatment comprising irradiation cancer cells using ionizing radiation. In a particular embodiment, exposing the cancer cells to a cancer treatment comprises applying ionizing radiation to the cancer cells cultured in the 3D scaffold.

Targeted cancer therapy is a cancer treatment that uses drugs to target specific genes and proteins that are involved in the growth and survival of cancer cells. Targeted therapy can affect the cancer cells themselves, but also the tissue environment that helps a cancer grow and survive, or it can target cells related to cancer growth like blood vessels.

Immunotherapy is a cancer treatment that is based on activating the immune system. Cell-based immunotherapies are effective for some cancers using immune effector cells, such as lymphocytes, macrophages, dendritic cells, natural killer cells, and cytotoxic T lymphocytes to defend the body against cancer by targeting abnormal antigens expressed on the surface of cancer cells. Such immunotherapies also include therapies involving engineered immune cells, such as chimeric antigen receptor (CAR) T cell therapy exposing the cancer cells to a cancer treatment comprises adding immune effector cells to the cancer cells cultured in the 3D scaffold.

In an embodiment, the method comprises analyzing the expression of at least one biomarker in the cancer cells cultured in the 3D scaffold prior to exposing the cancer cells to the cancer treatment. The method also comprises analyzing the expression of the at least one biomarker in the cancer cells cultured in the 3D scaffold after exposing the cancer cells to the cancer treatment. The method further comprises comparing the expression of the at least one biomarker in the cancer cells cultured in the 3D scaffold prior to exposing the cancer cells to the cancer treatment with the expression of the at least biomarker in the cancer cells cultured in the 3D scaffold after exposing the cancer cells to the cancer treatment. In this embodiment, the response of the cancer cells to the cancer treatment is determined based on the comparison.

In another embodiment, the method comprises analyzing the expression of at least one biomarker in the cancer cells cultured in a 3D scaffold after exposing the cancer cells to the cancer treatment. The method also comprises analyzing the expression of the at least one biomarker in the cancer cells cultured in a 3D scaffold without exposing the cancer cells to the cancer treatment. The method further comprises comparing the expression of the at least one biomarker in the cancer cells cultured in the 3D scaffold without exposing the cancer cells to the cancer treatment with the expression of the at least biomarker in the cancer cells cultured in the 3D scaffold after exposing the cancer cells to the cancer treatment. In this embodiment, the response of the cancer cells to the cancer treatment is determined based on the comparison.

The comparison of expressions of biomarkers can provide valuable diagnostic information of the population of cancer cells surviving the cancer treatment. For instance, the cancer treatment may cause a change in the populations of cancer cells cultured in the 3D scaffold, such as by mainly killing a highly proliferative population of cancer cells thereby leaving a more quiescent, dormant population of cancer cell remaining viable following the cancer treatment. The comparison in expression of biomarkers can thereby give, among others, such information relating to any changes in cancer cell populations and giving information of which cancer cell populations that are mainly affected by the cancer treatment and the characteristics of the cancer cells escaping or surviving the cancer treatment.

In an embodiment, the method comprises determining the viability of the cancer cells cultured in the 3D scaffold prior to exposing the cancer cells to the cancer treatment. The method also comprises determining the viability of the cancer cells cultured in the 3D scaffold after exposing the cancer cells to the cancer treatment. The method further comprises comparing the viability of the cancer cells cultured in the 3D scaffold prior to exposing the cancer cells to the cancer treatment with the viability of the cancer cells cultured in the 3D scaffold after exposing the cancer cells to the cancer treatment. The method additionally comprises determining the response of the cancer cells to the cancer treatment based on the comparison.

In another embodiment, the method comprises determining the viability of the cancer cells cultured in a 3D scaffold without exposing the cancer cells to the cancer treatment. The method also comprises determining the viability of the cancer cells cultured in a 3D scaffold after exposing the cancer cells to the cancer treatment. The method further comprises comparing the viability of the cancer cells cultured in the 3D scaffold without exposing the cancer cells to the cancer treatment with the viability of the cancer cells cultured in the 3D scaffold after exposing the cancer cells to the cancer treatment. The method additionally comprises determining the response of the cancer cells to the cancer treatment based on the comparison.

Viability as used herein is a measure of the proportion of live cells within a cell population. Cell viability may be assayed through measurement of, for instance, metabolic activity, ATP count, membrane integrity dyes, such as propidium iodide or 7-aminoactinomycin D (7-AAD), cell-impermeant fluorescent viability dyes, and/or enzymatic activity substrates.

In this embodiment, the 3D scaffold can be used as an in vitro model to monitor and assess the efficacy of the cancer treatment and where this information is of high value as a prediction of the efficacy of the cancer treatment when applied to a patient. Thus, if the cell viability is comparatively lower following the cancer treatment, the cancer treatment is determined to be effective, whereas if the cell viability after the cancer treatment is not significantly different from the cell viability prior to the cancer treatment, the cancer treatment is determined to be less effective for the subject and another cancer treatment should instead be investigated.

### EXAMPLES

### Example 1: Identification of Proteins that are Differentially Expressed in a Set of Decellularized Tumor Tissue Samples as Compared to a Set of Reference Samples

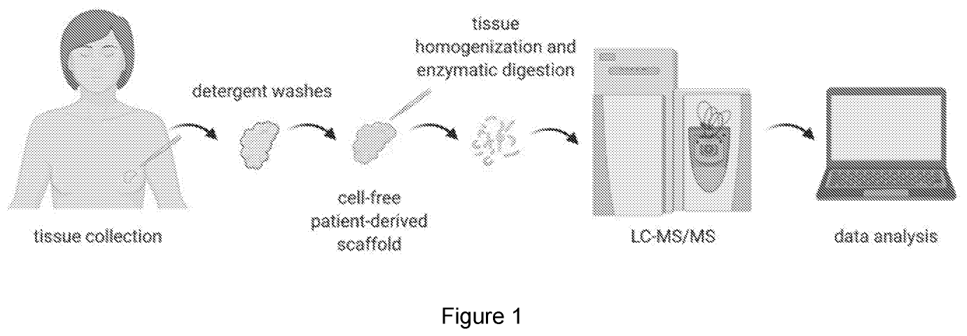

FIG. 1 schematically illustrates the workflow from tissue collection to data analysis performed in this Example.

### Patient Material

Frozen breast cancer tissues were collected from Sahlgrenska University Hospital biobank, and fresh cancer tissues were received directly after surgery from Sahlgrenska University Hospital Pathology and Diagnostics unit, Sweden. Patient consent to process patient material and data was waived for usage of biobanked materials by the Regional Ethics Committee in Gothenburg, while written consent was obtained from all patients donating fresh tissues. Biobank samples were collected from 1980 to 1999 in Sweden, and patients were monitored until endpoint of 2012 Nov. 15.

TABLE 1
Characteristics of cancer patients and their tumors
included in this study. IDC, invasive ductal carcinoma;
ILC, invasive lobular carcinoma. 1844 proteins
were identified in the decellularized tumor
tissue samples as compared to in reference tissue
(8 “normal/adjacent” tissue samples).

| | |
|---|---|
| Number of patients | 65 |
| Age median | 67 |
| Histologic type | |
| IDC | 43 |
| ILC | 16 |
| Other | 6 |
| Tumor grade | |
| Grade I | 21 |
| Grade II | 19 |
| Grade III | 24 |
| Missing | 1 |
| Disease recurrence | |
| Negative | 33 |
| Positive | 32 |
| Tumor size | |
| T ≤2 cm | 8 |
| T >2 cm | 54 |
| Missing | 3 |
| Estrogen receptor | |
| Positive | 55 |
| Negative | 9 |
| Missing | 1 |
| Progesterone receptor | |
| Positive | 44 |
| Negative | 19 |
| Missing | 2 |
| Lymph-node metastasis | |
| Positive | 32 |
| Negative | 23 |
| Missing | 10 |

### Patient-Derived Decellularized Tumor Tissue Sample Preparation

Frozen primary breast cancers (n=65) and adjacent tissue (n=8) were decellularized with lysis buffer containing 0.5 mM ethylenediaminetetraacetic acid (EDTA; Applichem), 3.5 mM sodium dodecyl sulfate (SDS; Applichem), 0.4 mM phenylmethanesulfonyl fluoride solution (PMSF; Merck), 3.07 mM sodium azide (G-Biosciences), and water. The decellularization was followed by a rinse with phosphate buffered saline (PBS; Medicago) supplemented with sodium azide, EDTA and PMSF for 15 minutes. After 72 hours, the decellularized tumor tissues were washed with water followed by a 24 hour wash with PBS to remove cellular debris. Decellularization was performed at 37° C., 175 rpm. Decellularized tumor tissues were then placed in storage solution containing sodium azide, EDTA and dH₂O, to preserve the tissue until analysis. Cellular removal was evaluated by random DNA assessment using Qubit DNA HS Assay (Invitrogen) according to manufacturer's instructions.

### Quantitative Proteomic Analysis and Mass Spectrometry Analysis.

Quantitative proteomics analysis was performed at the Proteomics Core Facility of Sahlgrenska Academy, University of Gothenburg. Cell pellets were homogenized using a FastPrep®-24 instrument (1.4 mm ceramic spheres, Matrix D (green); MP Biomedicals, OH, USA) for 4 repeated 40 seconds cycles at 6.5 m/s in 200 μl of the buffer containing 2% SDS and 50 mM triethylammonium bicarbonate (TEAB). Samples were centrifuged at 14,800 rpm for 5 min at RT (20-25° C.) and the supernatants were transferred to clean tubes. The lysis tubes were washed with 100 μl of the lysis buffer, centrifuged at 14,800 rpm for 20 min and the supernatants were combined with the corresponding lysates from the previous step. Lysates were filtered through the Corning Costar Spin-X 0.45 cellulose acetate centrifuge tube filter (MilliporeSigma, St. Louis, MO, USA), study 1) or centrifuged at 14,800 rpm for 20 min at 8° C. to remove fat and cell debris. Protein concentrations were determined using Pierce™ BCA Protein Assay Kit (Thermo Fischer Scientific, Waltham, MA, USA) and the Benchmark™ Plus microplate reader (Bio-Rad Laboratories, Hercules, CA, USA) with bovine serum albumin (BSA) solutions as standards. A representative reference pool to be included in each TMT-set was prepared by taking equal protein amount from all groups.

### Tryptic Digestion and Tandem Mass Tag (TMT) Labeling

For Tryptic digestion and Tandem Mass Tag (TMT) labeling, aliquots containing 20 μg (study 1) or 30 μg (study 2) of total protein were taken from each sample and the reference pool followed by reduction with DL-dithiothreitol (DTT, 100 mM final concentration) at 60° C. for 30 min. The reduced samples were processed using the modified filter-aided sample preparation (FASP) method (Wisniewski J R et. al. Nat Methods. 2009; 6 (5): 359-362). In short, reduced samples were diluted by 8 M urea, transferred onto Nanosep 30k Omega filters (Pall Corporation, Port Washington, NY, USA) and washed 2 times with 250 μl of 8 M urea. Alkylation of the cysteine residues was performed using 10 mM methyl methanethiosulfonate (MMTS) diluted in digestion buffer (0.5% sodium deoxycholate (SDC), 50 mM TEAB) for 30 min at room temperature and the filters were then repeatedly washed with digestion buffer. Trypsin (Pierce Trypsin Protease, MS Grade, Thermo Fisher Scientific) in digestion buffer was added in a ratio of 1:100 relative to total protein mass and the samples were incubated at 37° C. for 3 h; another portion of trypsin (1:100) was added and incubated overnight. The peptides were collected by centrifugation and labelled using Tandem Mass Tag (TMT) reagents (Thermo Fischer Scientific) according to the manufacturer's instructions. The labeled samples were combined into five (study 1) and eight (study 2) TMT sets, concentrated using vacuum centrifugation and SDC was removed by acidification with 10% triflouroacetic acid (TFA). The TMT sets were fractionated into 40 primary fractions by basic reversed-phase chromatography (bRP-LC) using a Dionex Ultimate 3000 UPLC system (Thermo Fischer Scientific). Peptide separations were performed using a reversed-phase XBridge BEH C18 column (3.5 μm, 3.0×150 mm, Waters Corporation) and a linear gradient from 3% to 40% solvent B over 17 min followed by an increase to 100% B over 5 min. Solvent A was 10 mM ammonium formate buffer at pH 10.00 and solvent B was 90% acetonitrile, 10% 10 mM ammonium formate at pH 10.00. The primary fractions were concatenated into final 20 fractions (1+21, 2+22, . . . 20+40), evaporated and reconstituted in 15 ul of 3% acetonitrile, 0.2% formic acid for nanoflow LC-MS analysis.

### LC-MS/MS Analysis

The fractions were analyzed on an Orbitrap Fusion Tribrid (study 1) or Fusion Lumos Tibrid (study 2) mass spectrometer interfaced with Easy-nLC 1200 liquid chromatography system (both Thermo Fisher Scientific). Peptides were trapped on an Acclaim Pepmap 100 C18 trap column (100 μm×2 cm, particle size 5 μm, Thermo Fischer Scientific) and separated on an analytical column (75 μm×30 cm, packed in-house with Reprosil-Pur C18, particle size 3 μm, Dr. Maisch, Ammerbuch, Germany) using a linear gradient from 5% to 35% B over 75 min followed by an increase to 100% B for 5 min, and 100% B for 10 min at a flow of 300 nL/min. Solvent A was 0.2% formic acid in water and solvent B was 80% acetonitrile, 0.2% formic acid. MS scans was performed at 120,000 resolution, m/z range 380-1380. The most abundant doubly or multiply charged precursors from the MS1 scans were isolated using the quadrupole with 0.7 m/z isolation window with a “top speed” cycle of 3 s and dynamic exclusion within 10 ppm during 45 s. The isolated precursors were fragmented by collision induced dissociation (CID) at 35% collision energy with the maximum injection time of 50 ms, and detected in the ion trap, followed by multinotch isolation of the top MS2 fragment ions, with m/z 400-1400, selected for fragmentation (MS3) by higher-energy collision dissociation (HCD) at 60% and detection in the Orbitrap at 50,000 resolution, m/z range 100-500.

### Proteomic Data Analysis

The data files were merged for each set for identification and relative quantification using Proteome Discoverer version 2.2 (study 1) or 2.4 (study 2) (Thermo Fisher Scientific). The database search was performed using the Mascot search engine v. 2.5.1 (Matrix Science, London, UK) against the Swiss-Prot Homo sapiens database (November 2017 (study 1) or June 2019 (study 2)) with MS peptide tolerance of 5 ppm and fragment ion tolerance of 0.6 Da. Tryptic peptides were accepted with no missed cleavages. Methionine oxidation was set as a variable modification, cysteine methylthiolation, tandem mass tag 6-plex (TMT-6) on lysine and peptide N-termini were set as fixed modifications. Percolator was used for peptide-spectrum match (PSM) validation with the strict false discovery rate (FDR) threshold of 1%. TMT reporter ions were identified in the MS3 high energy collision dissociation (HCD) spectra with 3 mmu mass tolerance, and the TMT reporter S/N values for each sample were normalized within Proteome Discoverer on the total peptide amount. Only the unique identified peptides were taken into account for the relative quantification and the proteins were filtered at 1% FDR.

### Statistical Methods

Student's t-test was used to distinguish proteins that were differentially expressed between the groups of tumors that came from patients with recurrence and without recurrence. Further, Mann-Whitney U, Kruskall-Wallis analyses as well as univariate, multivariable analyses were achieved by SPSS (IBM Logistics). Disease-free survival was assessed using Kaplan-Meier analyses with log-rank test. Group cut-offs were defined as quartiles or median of the entire populations. Panther tool Version 15.0 was used for protein classifications using GeneOnthology. Analysis was performed against Reactome version 73 on 22/09/2020.

### Results

The mass spectrometry analyses of patient-derived decellularized breast cancer samples identified in total 1844 proteins expressed in varying amounts in the different patient-derived decellularized breast cancer samples. In order to identify differences in the composition of the patient-derived decellularized breast cancer samples potentially associated with various clinical properties, the inventors related several common clinio-pathological features with the mass spectrometry data as presented in Table 2 below. The differences in protein presence in relation to clinical behaviors supports that the cancer microenvironment monitored via cell-free scaffolds, mirrors and influence cancer progression and patient outcome and that the identified proteins in Table 2 can be part of mediating aggressive properties.

TABLE 2

| | Number of proteins |
|---|---|
| | differentially expressed |
| ECM proteins in tumor vs reference tissue | 126 |
| High vs low grade tumors | 631 |
| Lymph-node metastasis + cancers | 52 |
| ER+ vs ER− | 69 |
| PR+ vs PR− | 72 |
| Lobular vs ductal cancer | 462 |
| Small (<2 cm) vs large tumors | 152 |
| Recurring vs non-recurring disease | 74 |

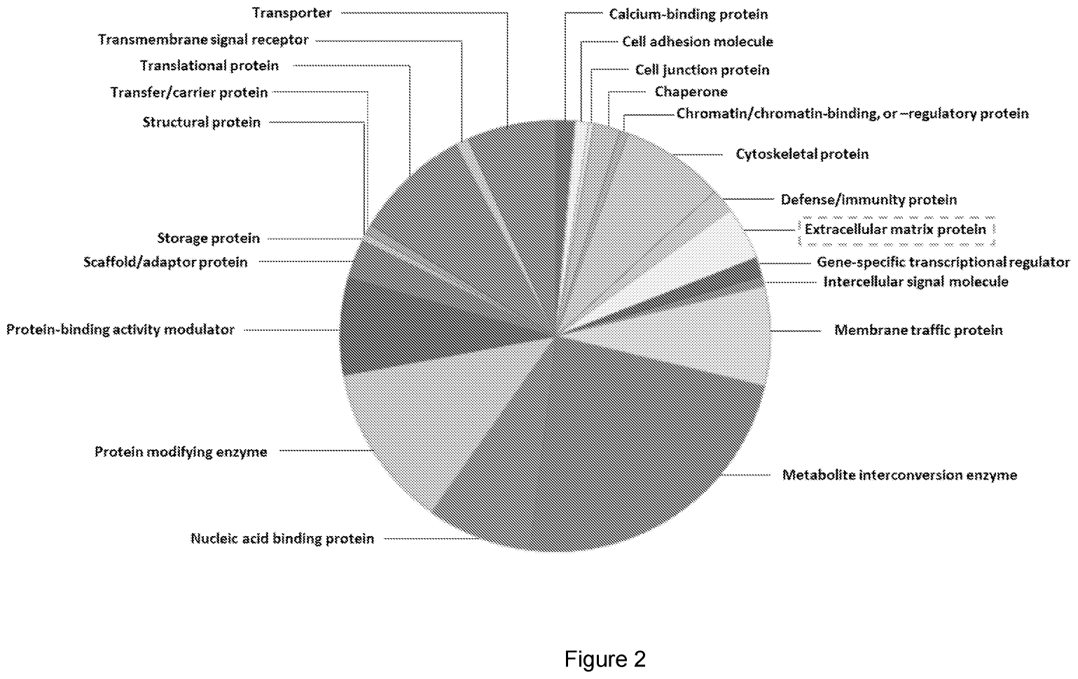

FIG. 2 illustrates protein classification according to PANTHER Protein Class. The pie chart illustrates percentage of types of proteins in the 1844 proteins detected in patient-derived decellularized tumor samples to each category against 1158 total number of Protein Class hits resulting from PANTHER classification analysis [2]. Noteworthy is that extracellular matrix (ECM) proteins only represent about 4% of the total number of proteins. This Example shows that patient-derived decellularized tumor tissue samples include several other proteins besides ECM. Hence, a 3D scaffold as produced by a bioink should thereby contain other protein components and not only ECM proteins if the 3D scaffold should mimic the tumor and its tumor properties.

### Example 2: PPI Network Construction and Analysis

Since proteins rarely perform biological functions independently, it is important to be aware of protein interactions by studying functional groups. A PPI network was established by the STRING app (http://apps.cytoscape.org/apps/stringapp) in Cytoscape software version 3.6.0. The software used the default parameters for analysis, and the connectivity degree of each node in the network was calculated by connectivity analysis. DEGs with a degree of connectivity ≥5 were defined as having high degrees of connectivity and were used to screen for core genes.

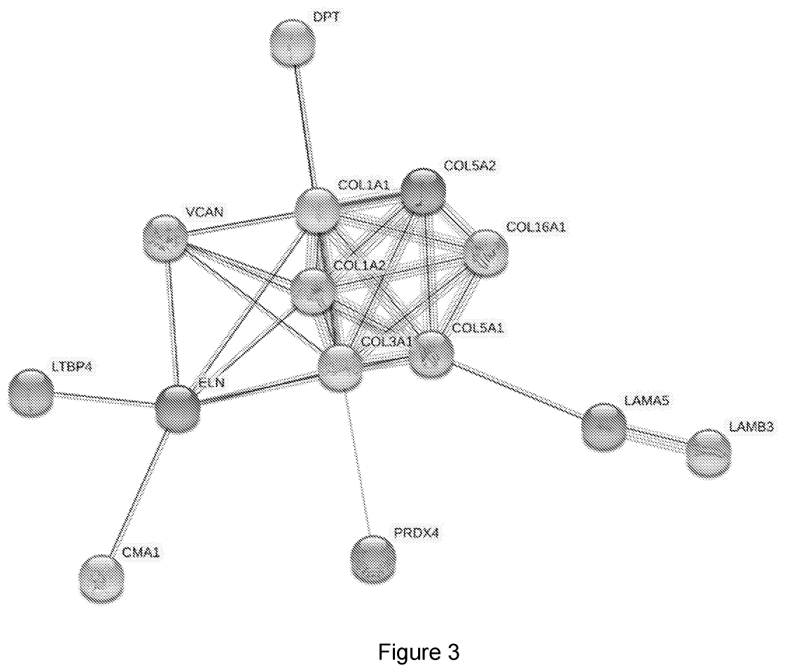

FIG. 3 illustrates the top 15 proteins showing the highest average protein contents in all analyzed patient-derived decellularized tumor samples. STRING was used to illustrate known or predicted protein-protein interactions (lines) for the top 15 proteins. This data exemplifies actual data from patient material identifying proteins that are present in all patient-derived decellularized tumor samples and which represent important building blocks for bioinks aimed at mimicking the in vivo microenvironment. The string analysis and presentation also illustrate that these proteins interact, and that they thereby have similar functions and relate to each other. Central proteins can also be identified having several interactions or being more central in clusters of interactions (COL1, COL3, COL5, ELN, VCAN). All, or some, of these proteins are advantageously included in a bioink.

### Example 3: Key Protein Identification

In order to identify key proteins that are likely to contribute to cancer progression and various clinical behaviors, different strategies as exemplified below were used. One approach was to identify proteins commonly expressed in all patient-derived decellularized tumor samples, as illustrated in FIG. 3 and Example 2, identifying the top 15 proteins, which were highly expressed in all patient-derived decellularized tumor samples. By also adding a string analysis, visualizing known or predicted protein interactions, it was possible to define clusters of proteins or ‘hub proteins’ that are potentially more important in mediating “common” behaviors in the patient-derived decellularized tumor samples. An example is the collagen cluster including COL3A1, COL5A1, COL16A1, COL5A2, COL1A1, COL1A2 as well as ELN that show several interactions (FIG. 3).

Another strategy to identify key proteins in patient-derived decellularized tumor samples, was to list proteins that are statistically significantly different in, for example, breast cancer samples from patients with or without disease recurrences (FIG. 2 and Table 2 in Example 1). As illustrated in FIG. 2, some proteins were highly expressed in patient-derived decellularized tumor samples from breast cancer with recurrences, whereas others were significantly lower in recurrences and vice versa for no recurrences. Proteins that are important for mediating clinical aggressiveness in the patient-derived decellularized tumor samples include those proteins with the highest difference in protein contents between no recurrences versus recurrences. Such proteins include KRT10, KRT2, DCD, DSC1 and KRT1. Hence, a bioink used to produce a 3D scaffold mimicking a recurrent tumor preferably includes at least one protein selected from the group consisting of KRT10, KRT2, DCD, DSC1 and KRT1.

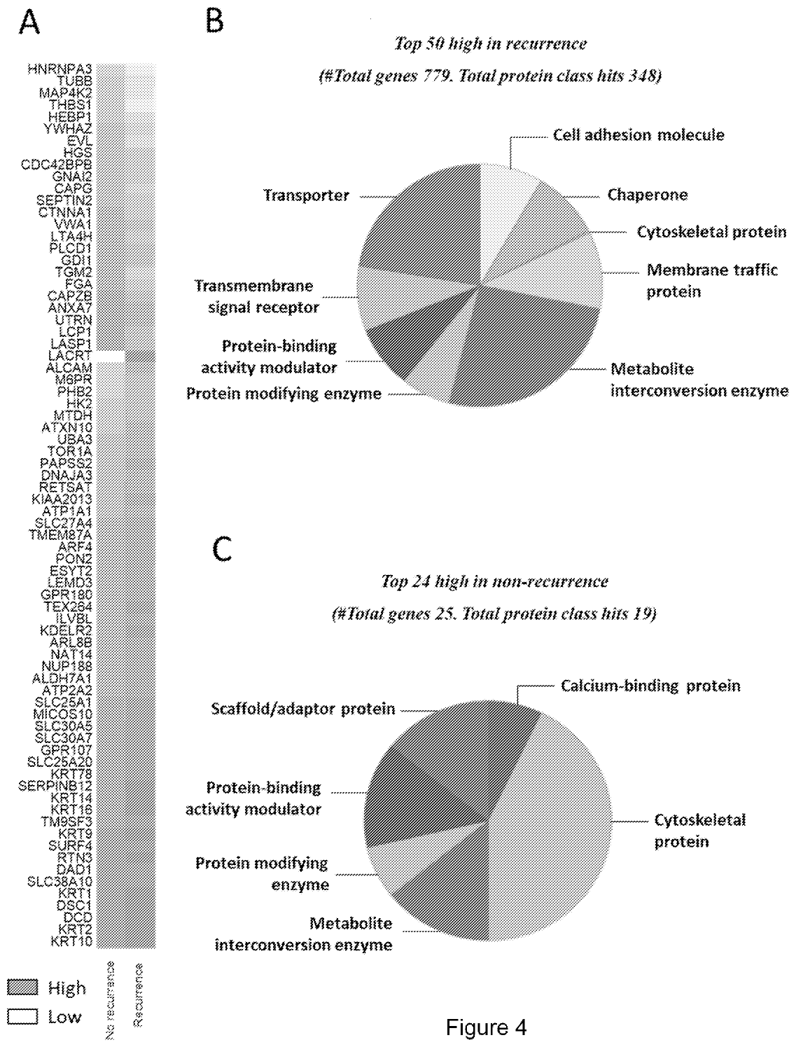

FIG. 4 illustrates top 50 proteins in patient-derived decellularized tumor samples associated with recurrence and top 24 proteins in patient-derived decellularized tumor samples associated with no recurrence. FIG. 4A is a heatmap representation of proteins significantly and differently expressed between patient-derived scaffolds from tumors derived from patients with or without disease recurrence. FIGS. 4B and 4C illustrate patient-derived scaffold protein classification according to PANTHER Protein Class. The pie charts illustrate the percentage of the various functional classification from patients with (FIG. 4B) recurrence or (FIG. 4C) no recurrence.

The results as presented in FIG. 4 illustrate that proteins are significantly differentially expressed in patient-derived decellularized tumor samples from patients with or without recurrences, thereby specifically indicating top proteins that may be included in a bioink in order to optimally mimic an aggressive microenvironment or a less aggressive microenvironment regarding the net effect of whether a cancer will spread or respond favorable to the treatments or not. The order of proteins in FIG. 4A also illustrates the significance level of the difference in expression and can guide in the selection of candidates for bioinks. FIGS. 4B and 4C further show that certain types of functions for the proteins differing between recurrent or not recurrent diseases are clearly distinct. Membrane trafficking proteins, transporters and transmembrane signaling receptors are clearly present in aggressive cell-free scaffold linked to recurrent diseases whereas cytoskeletal proteins and scaffold/adaptor proteins are more often present in less aggressive microenvironment with fewer disease recurrences,

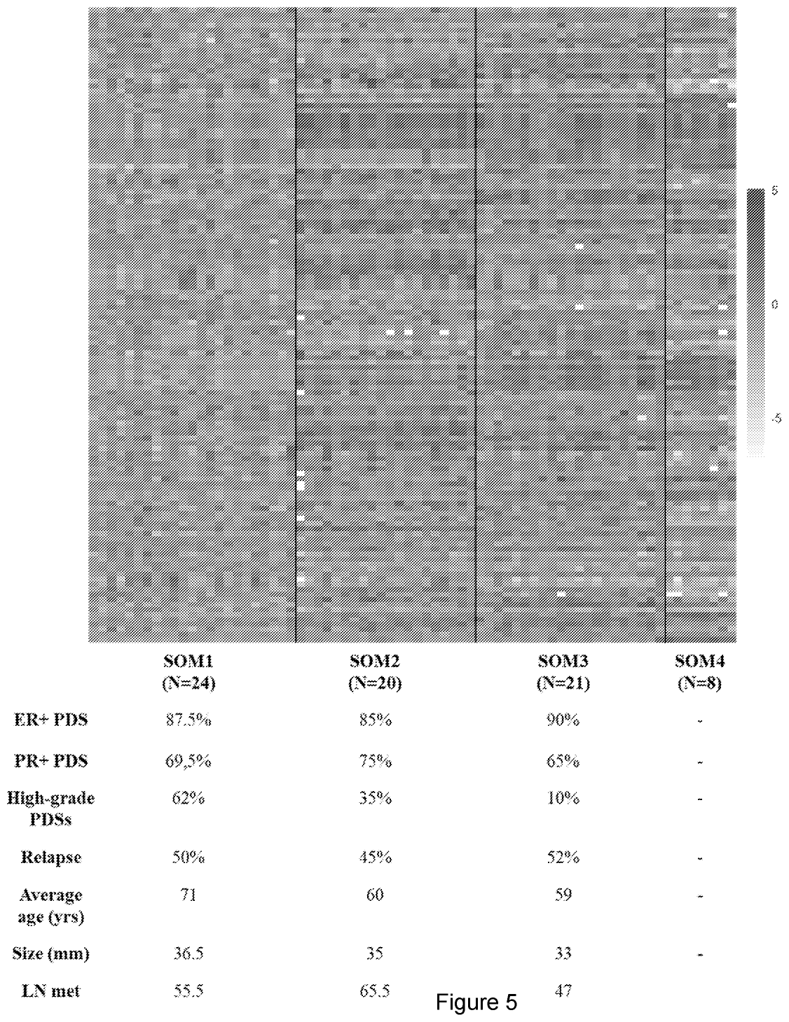

FIG. 5 illustrates that patient-derived decellularized tumor samples from tumors with high and low tumor grade can be subdivided based on their extracellular matrix (ECM) composition. Self-organizing map (SOM) analyses (GenEx, multid software) formed four groups of the 65 patient-derived decellularized tumor samples (PDSs) and 8 PDSs from adjacent tissues using the identified 126 extracellular matrix proteins (ECM as illustrated in a heat-map). The distribution of the SOM groups in relation to estrogen receptor (ER) and progesterone receptor (PR) status, size, high-grade, lymph node-metastasis and patients characteristics as disease-recurrence and age is also presented.

FIG. 5 shows that lower amounts of ECM proteins is associated with more aggressive tumors of a higher grade. SOM 1 showed in general lower amounts of the majority of all selected ECM proteins, whereas the other SOM groups had varying but higher presence of the ECM proteins. These data provide information of what proteins families that could be included in a bioink. ECM proteins can be used to model the aggressiveness towards less aggressive behaviors but are not the main components of a highly aggressive microenvironment regarding the percentage of different proteins present related to tumor grade.

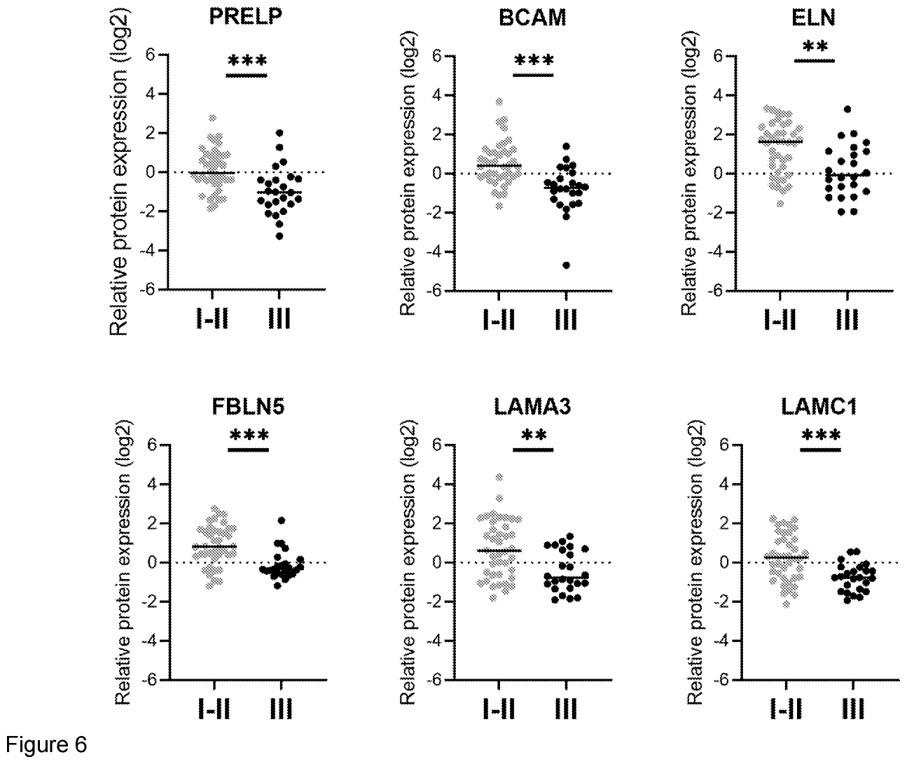

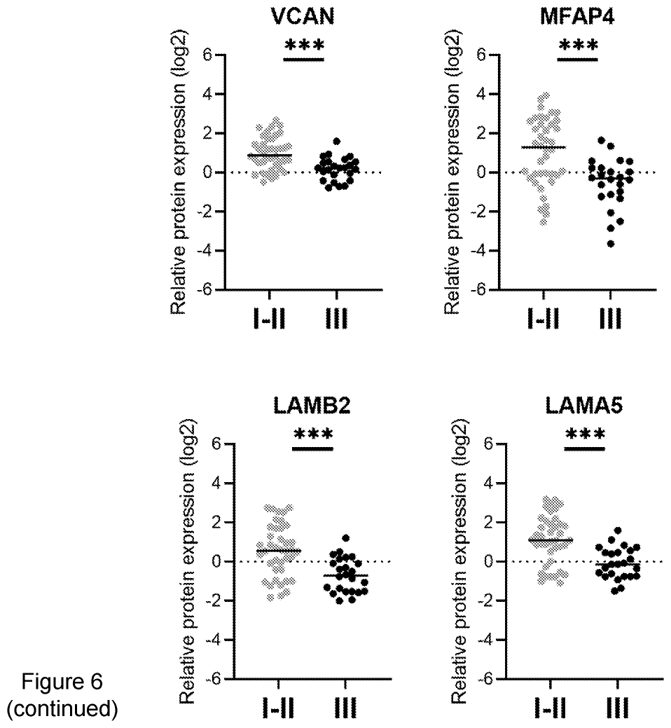

The inventors further identified that ECM proteins are more often present in patient-derived decellularized tumor samples from less aggressive breast cancer with lower grade. As illustrated in FIG. 6, several ECM proteins are significantly lower in high (III) grade breast cancer compared to grade I-II. This information could be used to influence the composition and grade resemblance of a bioink by adding ECM proteins, and specifically the identified proteins, to the bioink thereby modelling the aggressiveness of a certain cancer microenvironment. The results presented in FIG. 6 complement the results presented in FIG. 5 and show the specific proteins that significantly differ between high and low grade patient-derived decellularized tumor samples thereby guiding the choice of proteins that can be added to a bioink with some focus on less aggressive properties via mimicking a lower grade cancer microenvironment.

FIG. 7 illustrates that collagen abundance in the breast cancer microenvironment decreases with patient's age. In 10 out of 20 collagens patient-derived decellularized tumor samples collagen expression could be stratified based on patient's age. These data illustrate that certain collagens are significantly lower in patients of higher age when analyzing patient-derived decellularized tumor samples. The data can be used to model certain types of cancer microenvironments by adding the defined collagens to bioinks to create a 3D tumor model that is representative, for example, for younger cancer patient microenvironments. Hence, a similar approach could be used with the data presented in FIG. 7 and by adding the identified collagen proteins to a bioink it will be possible to mimic a certain age group of breast cancer.

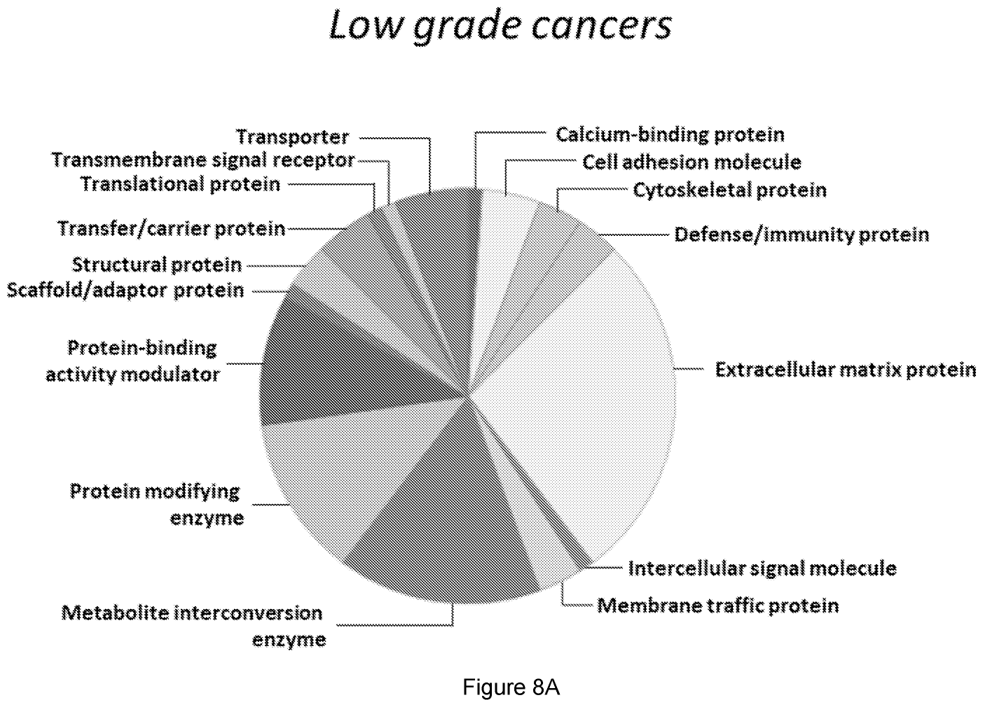

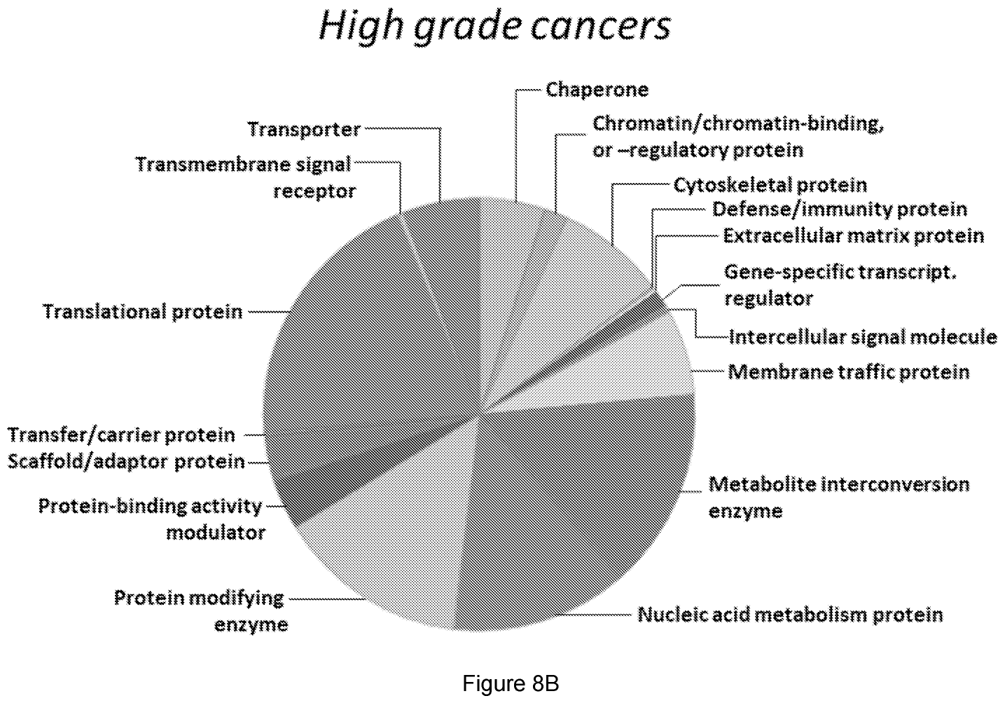

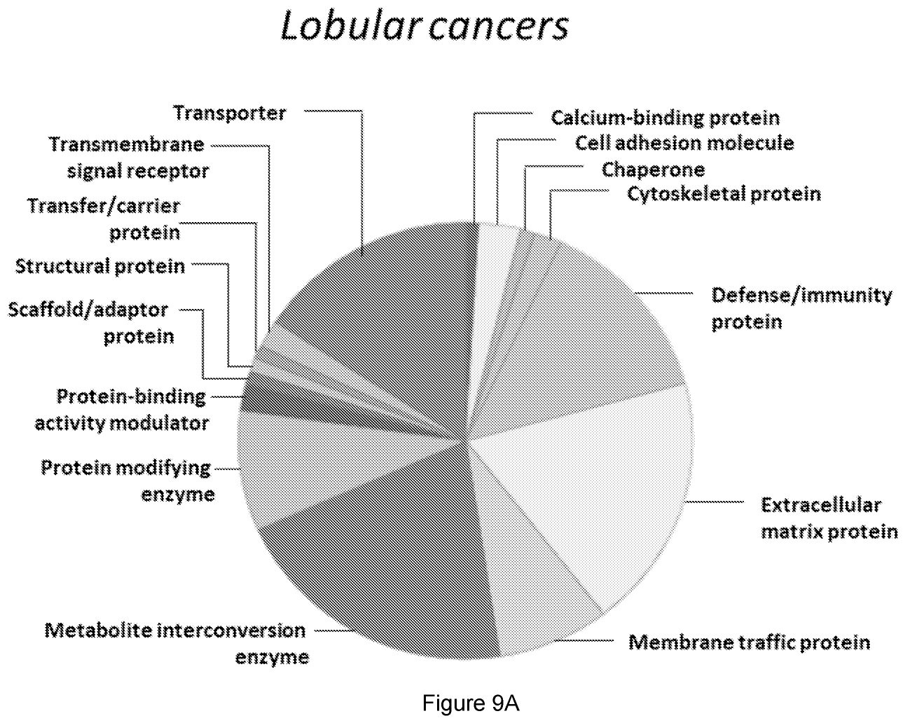

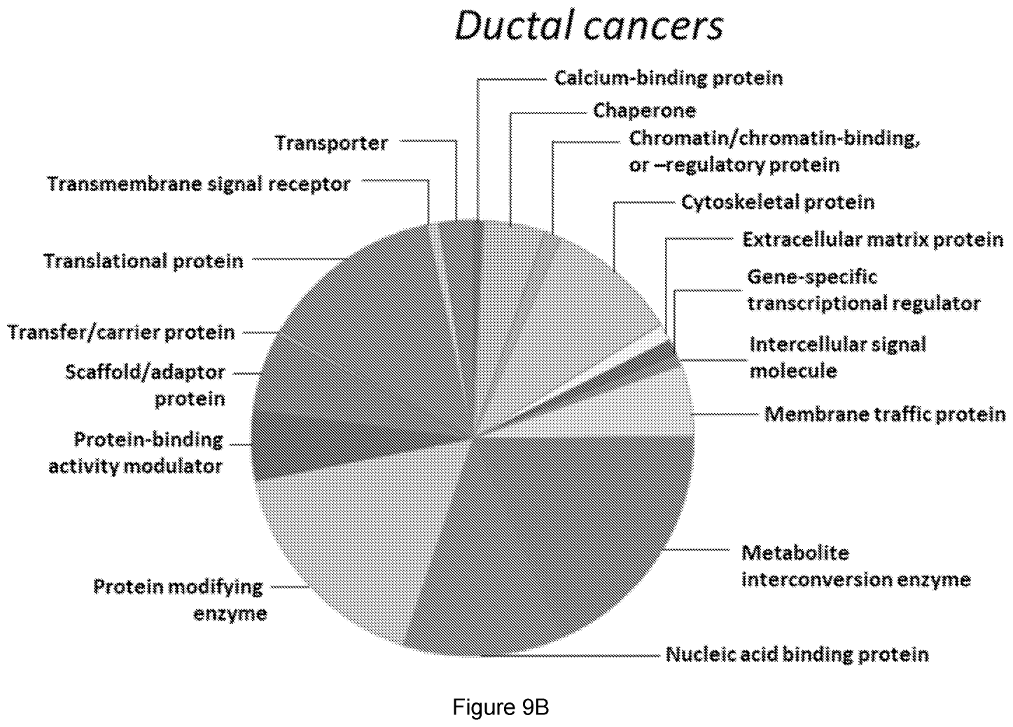

When clustering the proteins that are differently expressed in relation clinical properties it is also possible to identify certain functional families as illustrated in FIGS. 8A and 8B and FIGS. 9A and 9B for low grade versus high grade cancer, as well as for the subtypes of ductal versus lobular breast cancer. These data illustrate that, for example, ECM proteins and defense/immunity proteins are more frequent in lobular cancer in comparison to ductal cancer and if a bioink should mimic this subtype of cancer it is preferred to include these families of proteins in the bioink. It is also likely that ECM proteins and defense/immunity proteins are more critical for cancer growth in lobular cancer in comparison ductal cancer instead having more cytoskeletal proteins as an example.

FIGS. 8A and 8B illustrate that the functions for the proteins differing between low grade and high grade cancer microenvironments are clearly distinct. Low grade microenvironments have, for example, higher fraction of ECM proteins compared to high grade. By modelling the ECM protein content in a bioink, it is possible to mimic different grade microenvironments, thereby optimizing the composition of different bioinks depending on the purpose.

Correspondingly, FIGS. 9A and 9B illustrate that the functions for proteins differing between lobular and ductal cancer microenvironments are clearly distinct. Lobular cancers have, for example, higher fraction of ECM and defense/immunity proteins compared to ductal cancers, whereas ductal cancer have more metabolite interconversion enzyme and cytoskeletal proteins. By varying the proteins of the identified families in various bioinks it is possible to optimally mimic a certain type of microenvironment.

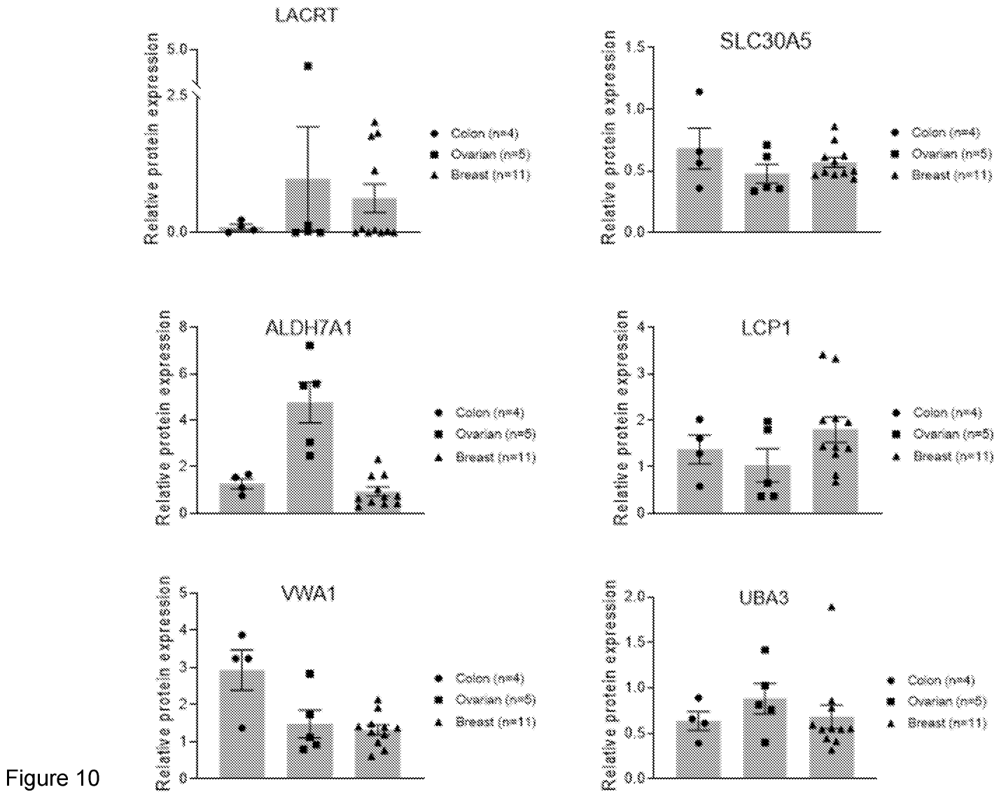

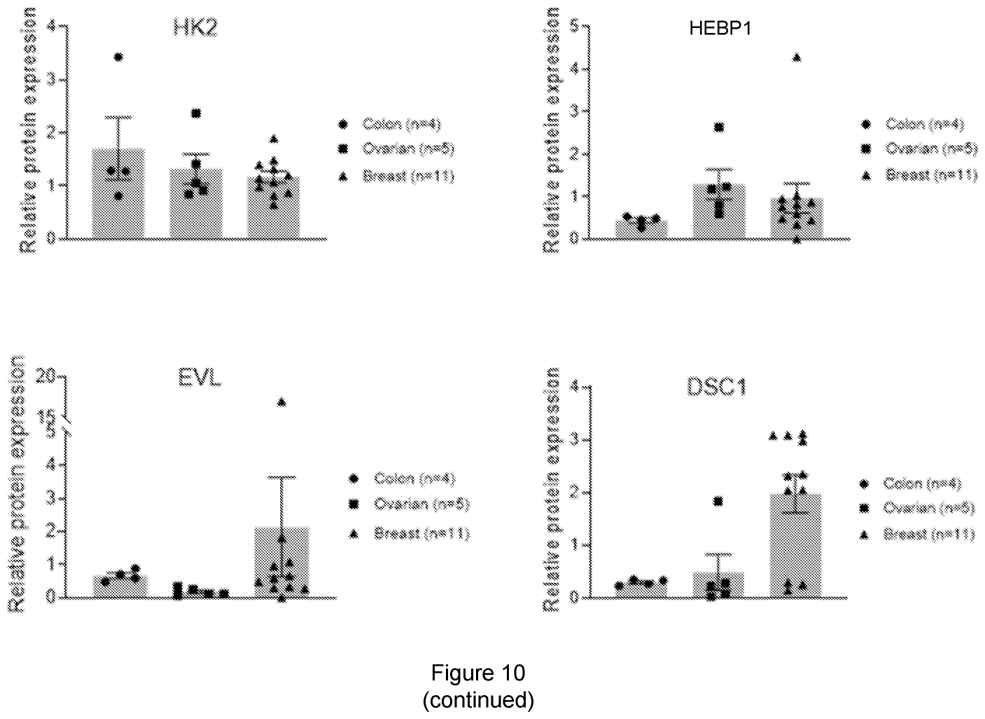

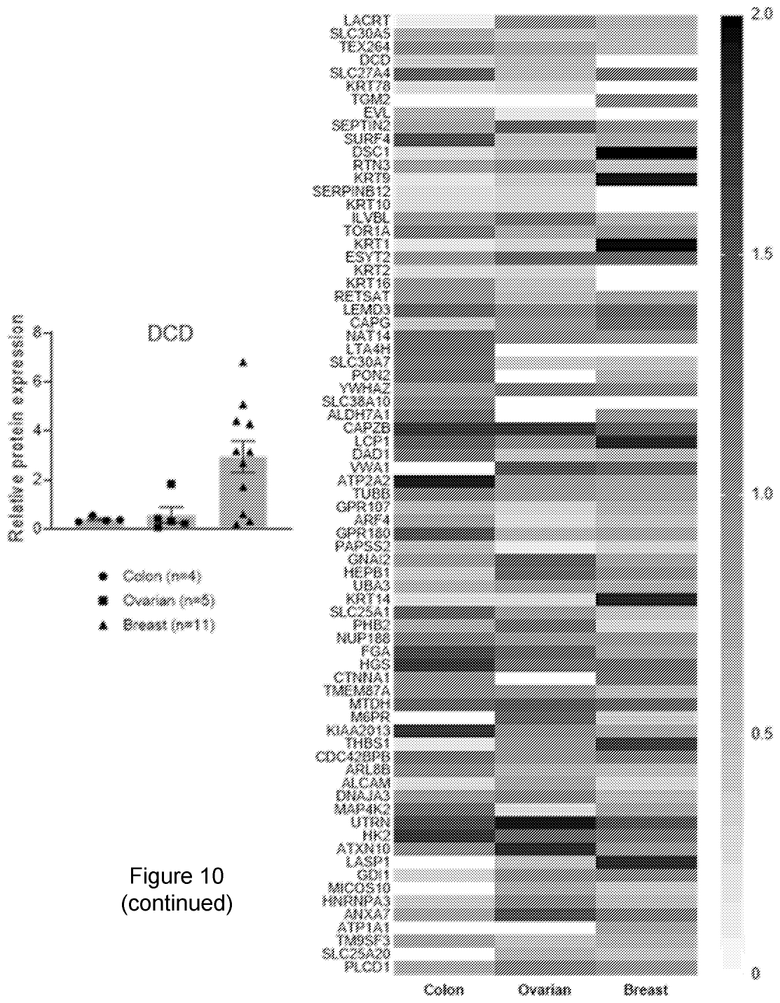

FIG. 10 illustrates that 73 of 74 proteins differing between recurrent and not recurrent breast cancer (FIG. 3) using patient-derived decellularized breast cancer samples are also present in patient-derived decellularized tumor samples from colon cancer and ovarian cancer in varying amounts. Selected proteins are presented in detail in histograms and the average expression of all proteins in the different cancer forms as heatmaps. These results can be used to guide the selection of proteins that could be present in a bioink representative for different cancer forms as indicated in the figure. It also illustrate that the proteins that are linked to disease recurrence in breast cancer are also present in other cancer forms and are therefore candidates for being included in bioinks representative of different types of cancer.

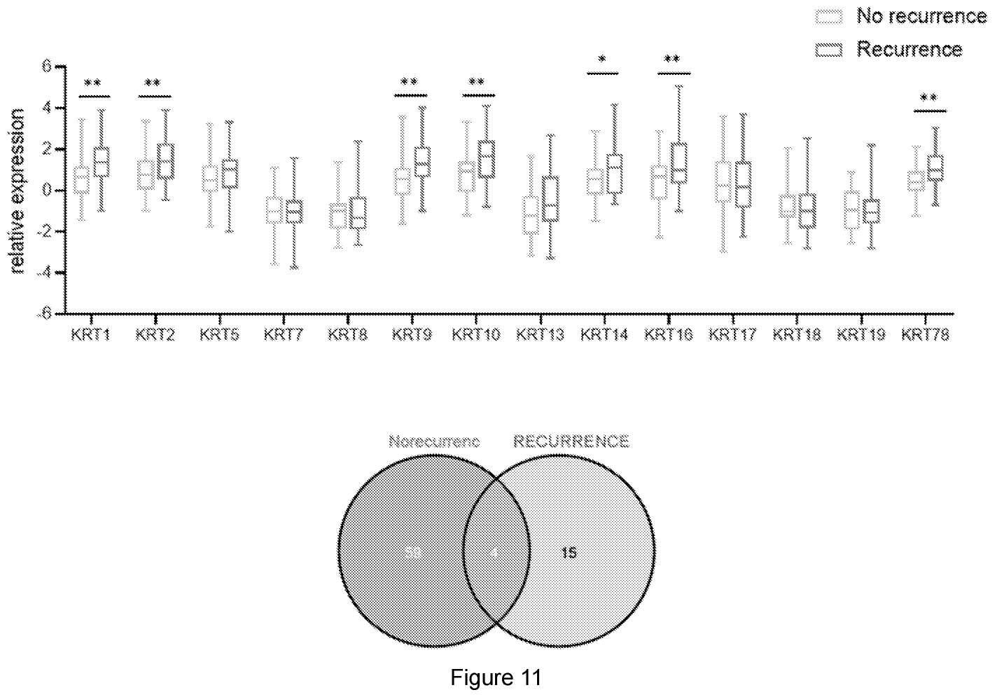

FIG. 11 illustrates that out of 74 differentially expressed proteins in patient-derived decellularized tissues from breast cancer with or without recurrences, 7 were identified as keratins. As illustrated in the figure using Log 2 expression of proteins in the keratin family, several keratins showed significantly higher protein contents in recurrent diseases. These data suggest that by varying the content of the defined keratins in a bioink, it is possible to model the aggressive behavior of the cancer microenvironments. It also supports that keratinization is an important process for cancer recurrences.

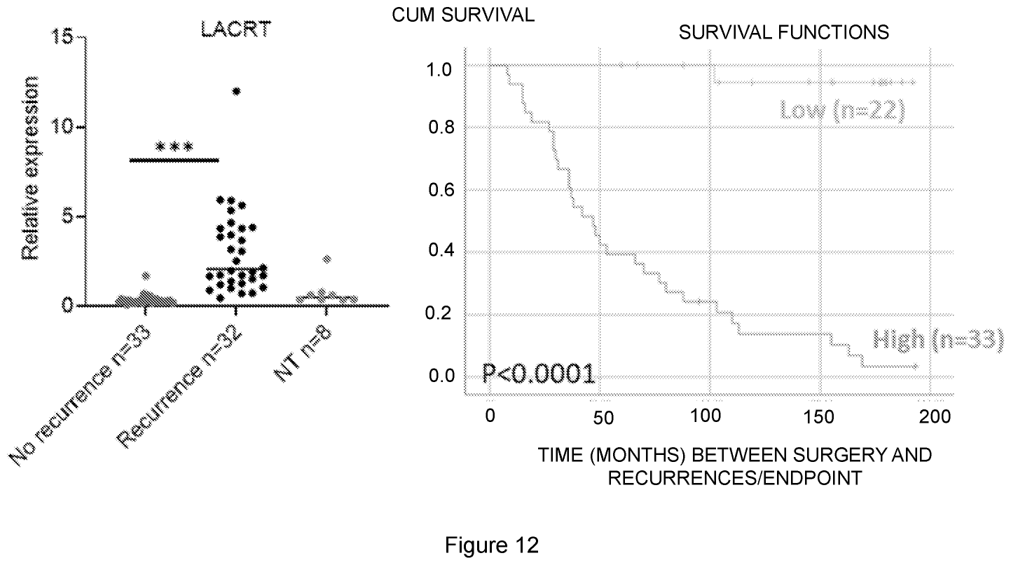

FIG. 12 illustrates that lacritin is expressed at higher levels in patient-derived decellularized tumor samples associated with disease recurrence compared to patient-derived decellularized tumor samples associated with no recurrence. These data suggest that lacritin presence in the cancer microenvironment is linked to recurrent breast cancer disease and can potentially be used in a bioink to model aggressiveness.

### Example 4: Construction Principles for 3D Scaffolds

This Example was designed to compare how different bioink base components and construction principles for 3D scaffolds affect cancer cells. MCF7 breast cancer cells were cultured for three weeks in either of the following: decellularized patient derived scaffolds, 3D printed alginate scaffolds, 3D Matrigel scaffolds, and electrospun nanofiber 3D scaffolds. The cancer cells from each type of 3D scaffold culture were then analyzed using qPCR and panels of important genes linked to key tumor biological processes were identified. Parallel 2D cultures were used as reference for the analysis of 3D-scaffold induced changes. PCA-analysis of 20 genes, which are known to be linked to cancer-related molecular and cellular processes revealed that all tested construction principles for 3D scaffolds induced a gene expression in the cancer cells, which was more similar to the gene expression in the cancer cells induced by the PDSs as compared to the gene expression in cells from control 2D cultures. The results obtained, using basic bioink components (nanocelluloce, alginate, and matrigel) without any added proteins, also revealed that electrospun nanocellulose 3D scaffolds replicated the tumor microenvironment to a higher degree as compared to 3D-printed alginate scaffolds, i.e., expression of the selected genes in cells cultured in electrospun 3D scaffolds was more similar to that obtained from cells cultured in PDS scaffolds as when compared to cells cultured in 3D printed alginate scaffolds. Using a heatmap illustration (FIG. 13A) with clustering of the different types of 3D scaffolds, based on the included gene expression analysis of the used MCF7 breast cancer cells, it was found that electrospun scaffolds clustered closer to PDS samples as compared to Matrigel and 3D-printed alginate cultures (FIG. 13B).

### Example 5: Click Chemistry for Coupling Selected Proteins to Basic Bioinks for 3D Scaffolds

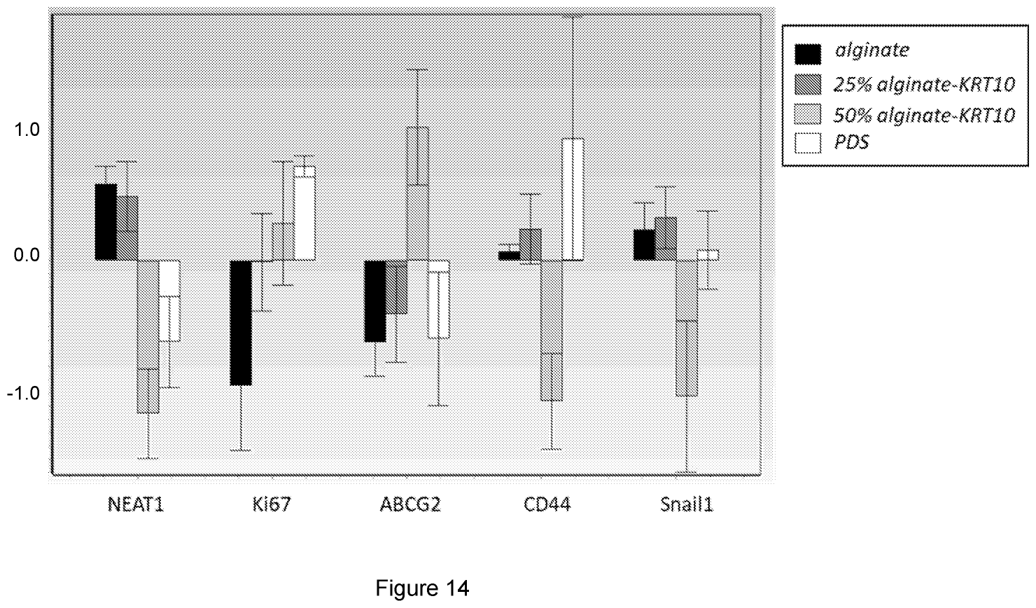

Alginate hydrogels are well-characterized, biologically inert materials that are used in many biomedical applications. Alginate, or similar inert materials, can be modified using click chemistry, which enables the production of derivatives with enhanced physical and chemical properties. In this experiment, alginate polymer chains were used to form covalently crosslinked click alginate hydrogels, incorporating proteins selected as components of a bioink to verify the effect these proteins as observed in cancer cells cultured in PDSs. In these experiments, MCF7 breast cancer cells were cultured for three weeks in either PDSs, in 3D printed alginate scaffolds, or in 3D printed alginate-KR10 scaffolds where the alginate had previously been crosslinked with keratin 10 (KRT10) by click-based coupling according to the following technique: The alginate was first modified via carbodiimide chemistry (EDC (1-ethyl-3-[3-dimethylaminopropyl] carbodiimide hydrochloride)/NHS (N-hydroxy sulfosuccinimide) to contain a triphenylalanine moiety and propargyl moiety. The triphenylalanine gives the bioink a shear thinning attribute that improves filament dimensions during construction of 3D scaffolds. The propargyl moiety allows an array of azide-containing proteins and peptides to be conjugated to the bioink. Keratin 10 (KRT10) was first modified to contain an azide moiety by EDC/NHS coupling and then it was covalently attached to the alginate via Huisgen's azide-alkyne cycloaddition click chemistry. The copper sulfate and ascorbic acid were removed by dialysis and the functionalized alginate was lyophilized prior to being redissolved for construction of 3D scaffolds by 3D printing. Before 3D printing the alginate-KR10, it was diluted so that the final alginate-KR10 concentration was either 25% or 50%. Several genes were investigated in the cancer cells after culture in the different control and KR10-modified 3D scaffolds and in a panel of 20 selected important genes linked to key tumor biological processes, 5 genes were found to be significantly different when comparing cells grown in the different type scaffolds, see FIG. 14. These results, thus, clearly illustrate a biological effect of the click-chemistry modification of the alginate with a selected protein for 3D based growth of cancer cells. Values for each of the genes were normalized to the same gene in MCF7 cancer cells grown in 2D cultures.

### Example 6: Identification of Cancer Treatment by Culturing Cancer Cells in 3D Scaffolds Manufactured Using Bioink of the Current Invention

Cancer drugs, such as immune targeting cancer drugs, can be identified and validated by culturing cancer cells in 3D scaffolds manufactured using any bioink of the current invention. Key proteins, such as selected immune proteins, are linked to a basic bioink, such as alginate or nanocellulose, and the resulting optimized bioink is used for the construction of 3D scaffolds (growth substrates) by, for instance 3D printing or electrospinning. The manufacturing technique is preferably selected for creating a 3D scaffold as similar to human tumor as possible. Cancer cells of any selected cancer type, such as breast cancer cells, colon cancer cells, ovarian cancer cells, melanoma cancer cells, or pancreas cancer cells, are grown in the 3D scaffold, which has been optimized to mimic selected tumor and clinical patient properties. Variations in the effects, such as gene expression and/or functional effects, after growth of the cancer cells with and without addition of the cancer drugs is measured using standard techniques, such as RNA sequencing and functional readouts, including, but not limited to, migration distance or viability. The biological effect of the treatment on the cancer cells growing in the optimized human tumor mimicking growth conditions is then analyzed and compared to non-treated cancer cells and the results are indicative of whether a cancer drug had an effect or not and also on how it worked.

As an illustrative example, breast cancer cells are grown in a 3D scaffold manufactured by electrospinning using a bioink based on nanocellulose, which has been linked to at least one protein to induce gene expression in the cancer cells similar to the gene expression in recurring breast cancer tumors. The at least one protein, which has been linked to the nanocellulose, can, for instance, be selected among KRT10, DSC10, SERPINB12, including any combination thereof. Cultures of breast cancer cells represent in vitro models of recurring tumors and they are subjected to a plurality of test compounds (one test compound per culture). At a specific time point after treatment, cancer cells are analyzed for gene expression associated with specific molecular properties including viability, migration/infiltration, proliferation, immune markers, stem cell properties etc., and compared to the gene expression in cancer cells of control cultures, which have not been subjected to treatment. The results indicate whether a test compound will be efficient as a treatment or not.

### Example 7: K-Means Clustering Analysis of ECM Proteins in Relation to all Proteins Identified in the PDS Scaffolds

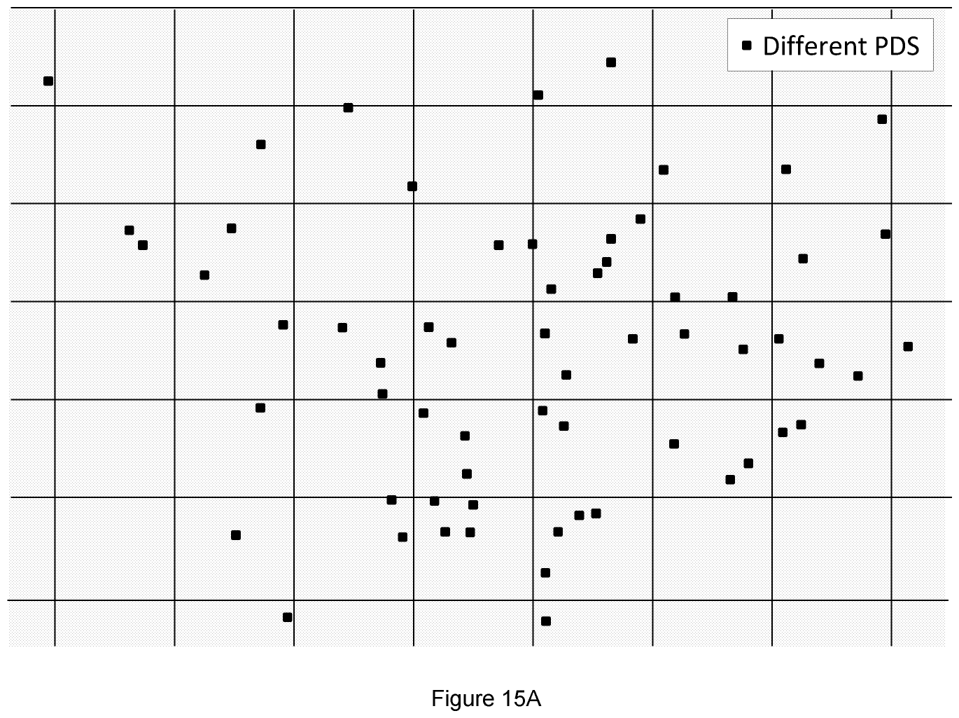

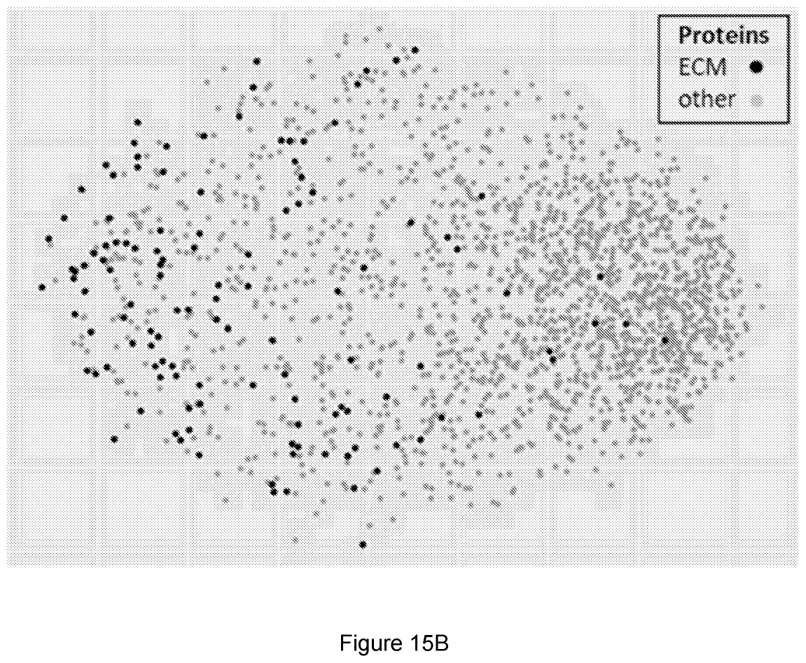

K-means clustering is a powerful unsupervised machine learning algorithm that can solve complex problems. Here, the algorithm was used to cluster proteins identified as ECM proteins based on tumor and on patient properties. The results of this method illustrate how various proteins present in the 3D scaffolds affect the positioning and clustering of PDSs (FIG. 15A) and proteins (ECM proteins vs all proteins; FIG. 15B). The relationship between ECM proteins and all proteins present in the PDSs (n=1844) were related to breast cancer features, patient information and SOM-groups (clusters). The data obtained indicate that PDSs comprising more ECM proteins are clearly different from PDSs comprising less ECM proteins. In addition, when defining clusters of similar PDSs using SOM groups based on either ECM-related proteins or based on all proteins, significant differences in the links to clinical properties were observed (Table 3). This suggests that ECM-related proteins alone for producing scaffolds are not sufficient to cover the broad spectrum of clinical behaviors and tumor properties. Thus, all proteins in the PDSs are required in the analysis (including, but not limited to ECM proteins) in order to optimally create a combination of proteins in the artificial 3D scaffolds.

TABLE 3

| Tumor or | ECM- | ECM- | ECM- | | All- | All | All | |
|---|---|---|---|---|---|---|---|---|
| clinical | cluster | cluster | cluster | p- | cluster | cluster | cluster | p- |
| property | 1 | 2 | 3 | value | 1 | 2 | 3 | value |
| Grade I-II | 11 | 8 | 18 | | 9 | 13 | 5 | |
| Grade III | 7 | 16 | 2 | 0.001* | 1 | 13 | 11 | 0.087 |
| ductal | 4 | 4 | 9 | | 4 | 20 | 17 | |
| lobular | 13 | 17 | 11 | | 7 | 3 | 7 | |
| other | 1 | 3 | 1 | 0.301 | 0 | 3 | 2 | 0.01* |
| <2 cm | 2 | 1 | 4 | | 4 | 1 | 2 | |
| 2-5 cm | 11 | 17 | 16 | | 7 | 17 | 20 | |
| >5 cm | 11 | 3 | 1 | | 0 | 5 | 3 | |
| edema | 0 | 2 | 0 | 0.232 | 0 | 2 | 0 | 0.04* |
| Age 36-51 | 6 | 1 | 9 | | 6 | 6 | 4 | |
| Age 52-69 | 6 | 7 | 4 | | 2 | 7 | 8 | |
| Age 70-86 | 6 | 16 | 8 | 0.026* | 3 | 13 | 14 | 0.17 |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

ANNEX A - table of gene names and protein names

| GENE NAME | PROTEIN NAME |
|---|---|
| ALCAM | Activated Leukocyte Cell Adhesion Molecule |
| ALDH7A1 | Alpha-aminoadipic semialdehyde dehydrogenase |
| ANXA7 | Annexin A7 |
| ARF4 | ADP-ribosylation factor 4 |
| ARL8B | ADP-ribosylation factor-like protein 8B |
| ATP1A1 | Sodium/potassium-transporting ATPase subunit alpha-1 |
| ATP2A2 | Protein transport protein Sec23B |
| ATXN10 | Ataxin-10 |
| BCAM | Basal cell adhesion molecule |
| CAPG | Macrophage-capping protein |
| CAPZB | F-actin-capping protein subunit beta |
| CDC42BPB | Serine/threonine-protein kinase MRCK beta |
| CMA1 | Chymase |
| COL1A1 | Collagen alpha-1(I) chain |
| COL1A2 | Collagen alpha-2(I) chain |
| COL3A1 | Collagen alpha-1(III) chain |
| COL4A1 | Collagen alpha-1(IV) chain |
| COL4A2 | Collagen alpha-2(IV) chain |
| COL5A1 | Collagen alpha-1(V) chain |
| COL5A2 | Collagen alpha-2(V) chain |
| COL6A1 | Collagen alpha-1(VI) chain |
| COL6A2 | Collagen alpha-2(VI) chain |
| COL6A3 | Collagen alpha-3(VI) chain |
| COL6A6 | Collagen alpha-6(VI) chain |
| COL7A1 | Collagen alpha-1(VII) chain |
| COL8A1 | Collagen alpha-1(VIII) chain |
| COL10A1 | Collagen alpha-1(X) chain |
| COL11A1 | Collagen alpha-1(XI) chain |
| COL12A1 | Collagen alpha-1(XII) chain |
| COL14A1 | Collagen alpha-1(XIV) chain |
| COL15A1 | Collagen alpha-1(XV) chain |
| COL16A1 | Collagen alpha-1(XVI) chain |
| COL18A1 | Collagen alpha-1(XVIII) chain |
| CTNNA1 | Catenin alpha-1 |
| DAD1 | Dolichyl-diphosphooligosaccharide -- protein glycosyltransferase subunit |
| | DAD1 |
| DCD | Dermcidin |
| DNAJA3 | DnaJ homolog subfamily A member 3, mitochondrial |
| DPT | Dermatopontin |
| DSC1 | Desmocollin-1 |
| ELN | Elastin |
| ESYT2 | Extended synaptotagmin-2 |
| EVL | Ena/VASP-like protein |
| FBLN5 | Fibulin-5 |
| FGA | Fibrinogen alpha chain |
| GDI1 | Rab GDP dissociation inhibitor alpha |
| GNAI2 | Guanine nucleotide-binding protein G(i) subunit alpha-2 |
| GPR107 | G Protein-Coupled Receptor 107 |
| GPR180 | Integral membrane protein GPR180 |
| HEBP1 | Heme Binding Protein 1 |
| HGS | Hepatocyte growth factor-regulated tyrosine kinase substrate |
| HK2 | Hexokinase-2 |
| HNRNPA3 | Heterogeneous nuclear ribonucleoprotein A3 |
| ILVBL | Acetolactate synthase-like protein |
| KDELR2 | ER lumen protein-retaining receptor 2 |
| KIAA2013 | Uncharacterized protein KIAA2013 |
| KRT1 | Keratin, type II cytoskeletal 1 |
| KRT2 | Keratin, type II cytoskeletal 2 epidermal |
| KRT5 | Keratin, type II cytoskeletal 5 |
| KRT7 | Keratin, type II cytoskeletal 7 |
| KRT9 | Keratin, type I cytoskeletal 9 |
| KRT10 | Keratin, type I cytoskeletal 10 |
| KRT13 | Keratin, type I cytoskeletal 13 |
| KRT14 | Keratin, type I cytoskeletal 14 |
| KRT16 | Keratin, type I cytoskeletal 16 |
| KRT17 | Keratin, type I cytoskeletal 17 |
| KRT18 | Keratin, type I cytoskeletal 18 |
| KRT19 | Keratin, type I cytoskeletal 19 |
| KRT78 | Keratin, type II cytoskeletal 78 |
| LACRT | Extracellular glycoprotein lacritin |
| LAMA3 | Laminin subunit alpha-3 |
| LAMA5 | Laminin subunit alpha-5 |
| LAMB2 | Laminin subunit beta-2 |
| LAMB3 | Laminin subunit beta-3 |
| LAMC1 | Laminin subunit gamma-1 |
| LASP1 | LIM and SH3 domain protein 1 |
| LCP1 | Plastin-2 |
| LEMD3 | Inner nuclear membrane protein Man1 |
| LTA4H | Leukotriene A-4 hydrolase |
| LTBP4 | Latent-transforming growth factor beta-binding protein 4 |
| M6PR | Cation-dependent mannose-6-phosphate receptor |
| MAP4K2 | Mitogen-Activated Protein Kinase Kinase Kinase Kinase 2 |
| MFAP4 | Microfibril-associated glycoprotein 4 |
| MICOS10 | Mitochondrial Contact Site And Cristae Organizing System Subunit 10 |
| MTDH | Protein LYRIC |
| NAT14 | N-acetyltransferase 14 |
| NUP188 | Nucleoporin NUP188 homolog |
| PAPSS2 | Bifunctional 3′-phosphoadenosine 5′-phosphosulfate synthase 2 |
| PHB2 | Prohibitin-2 |
| PLCD1 | 1-phosphatidylinositol 4,5-bisphosphate phosphodiesterase delta-1 |
| PON2 | Serum paraoxonase/arylesterase 2 |
| PRDX4 | Peroxiredoxin-4 |
| PRELP | Prolargin |
| RETSAT | All-trans-retinol 13,14-reductase |
| RTN3 | Reticulon-3 |
| SEPTIN2 | Septin-2 |
| SERPINB12 | Serpin B12 |
| SLC25A1 | Tricarboxylate transport protein, mitochondrial |
| SLC25A20 | Mitochondrial carnitine/acylcarnitine carrier protein |
| SLC27A4 | Long-chain fatty acid transport protein 4 |
| SLC30A5 | Zinc transporter 5 |
| SLC30A7 | Zinc transporter 7 |
| SLC38A10 | Putative sodium-coupled neutral amino acid transporter 10 |
| SURF4 | Surfeit locus protein 4 |
| TEX264 | Testis-expressed protein 264 |
| TGM2 | Protein-glutamine gamma-glutamyltransferase 2 |
| THBS1 | Thrombospondin 1 |
| TM9SF3 | Transmembrane 9 superfamily member 3 |
| TMEM87A | Transmembrane protein 87A |
| TOR1A | Torsin-1A |
| TUBB | Tubulin beta chain |
| UBA3 | NEDD8-activating enzyme E1 catalytic subunit |
| UTRN | Utrophin |
| VCAN | Versican core protein |
| VWA1 | von Willebrand factor A domain-containing protein 1 |
| YWHAZ | 14-3-3 protein zeta/delta |

1.-26. (canceled)

27. A method for producing a bioink, the method comprising:
analyzing a protein composition of a decellularized tumor tissue or tissues obtained from at least one tumor characterized by at least one defined tumor property selected from the group consisting of tumor grade, tumor size, cancer type or subtype, estrogen receptor (ER) status, progesterone receptor (PR) status, recurrence, and any combination thereof;
analyzing a protein composition of a reference decellularized tissue or tissues obtained from a reference tissue or tissues;
selecting at least one protein that is differentially expressed in the decellularized tumor tissue or tissues as compared to the reference tissue or tissues as determined based on the analyses; and
producing a bioink comprising the selected at least one protein, or a domain thereof, and configured for production of a three-dimensional (3D) scaffold mimicking a tumor characterized by the at least one defined tumor property.

28. The method according to claim 27, wherein the reference tissue or tissues is or are a non-tumor tissue or tissues.

29. The method according to claim 28, the reference tissue or tissues is or are a non-tumor tissue or tissues present adjacent to a respective tumor in a respective patient from which the at least one tumor is obtained.

30. The method according to claim 27, wherein the reference tissue or tissues is or are a reference tumor or tumors not characterized by the at least one defined tumor property.

31. The method according to claim 27, wherein
the at least one tumor is at least one grade III tumor and the reference tissue or tissues is or are a grade I or II tumor or tumors;
the at least one tumor is one of an invasive ductal carcinoma (IDC) and invasive lobular carcinoma (ILC) and the reference tissue or tissues is or are the other of IDS and ILC;
the at least one tumor is one of an ERα-negative tumor and an ERα-positive tumor and the reference tissue or tissues is or are the other of ERα-negative tumor and ERα-positive tumor;
the at least one tumor is one of an PR-negative tumor and a PR-positive tumor and the reference tissue or tissues is or are the other of PRα-negative tumor and PR-positive tumor;
the at least one tumor is at least one tumor having a size or volume larger than a threshold size or volume and the reference tissue or tissues is or are a tumor or tumors having a size/volume smaller than the threshold size or volume; and/or
the at least one tumor is at least one malignant tumor and the reference tissue or tissues is or are a non-malignant tumor or tumors.

32. The method according to claim 27, further comprising decellularizing tumor tissue obtained from at least one solid tumor to form the decellularized tumor tissue or tissues, wherein the at least one solid tumor is selected from the group consisting of at least one breast cancer tumor, at least one colon cancer tumor, at least one ovarian cancer tumor, at least one lung cancer tumor, and at least one pancreatic cancer tumor.

33. The method according to claim 27, wherein producing the bioink comprises producing the bioink comprising i) the selected at least one protein, or the domain thereof, and iia) at least one extracellular matrix (ECM) protein, or a structural domain thereof, and/or iib) at least one keratin, or a structural domain thereof, and/or iic) at least one lacritin, or a domain thereof.

34. The method according to claim 27, further comprising selecting at least one bioink base component based on a production method used to produce the 3D scaffold using the bioink, wherein producing the bioink comprises producing the bioink comprising the selected at least one protein, or the domain thereof, and the selected at least one bioink base component using the production method to form the 3D scaffold mimicking a tumor characterized by the at least one defined tumor property.

35. A method for producing a three dimensional (3D) scaffold comprising:
producing a bioink according to claim 27; and
producing the 3D scaffold using the bioink.

36. The method according to claim 36, wherein producing the 3D scaffold comprises bioprinting the bioink by a 3D printer or electrospinning the bioink to produce the 3D scaffold.

37. A bioink comprising at least one protein, or a domain thereof, which is differentially expressed in a decellularized tumor tissue or tissues as compared to a reference decellularized tissue or tissues, wherein
the decellularized tumor tissue or tissues is or are obtained from at least one tumor characterized by at least one defined tumor property selected from the group consisting of tumor grade, tumor size, cancer type or subtype, estrogen receptor (ER) status, progesterone receptor (PR) status, recurrence, and any combination thereof;
the reference decellularized tissue or tissues is or are obtained from a non-tumor tissue or tissue or from a reference tumor or tumors not characterized by the at least one defined tumor property; and
the at least one protein is selected by:
analyzing a protein composition of the decellularized tumor tissue or tissues;
analyzing a protein composition of the reference decellularized tissue or tissues; and
selecting at least one protein that is differentially expressed in the decellularized tumor tissue or tissues as compared to the reference tissue or tissues as determined based on the analyses.

38. The bioink according to claim 37, wherein
the at least one tumor property is recurrence; and
the at least one protein is selected from the group consisting of KRT1, KRT2, KRT10, DCD, DSC1, and any combination thereof.

39. The bioink according to claim 37, wherein
the at least one tumor property is recurrence; and
the at least one protein is selected from the group consisting of KRT1, KRT2, KRT9, KRT10, KRT14, KRT16, KRT78 and any combination thereof.

40. The bioink according to claim 37, wherein
the at least one tumor property is recurrence; and
the at least one protein is LACRT.

41. The bioink according to claim 37, wherein
the at least one tumor property is tumor grade; and
the at least one protein is selected from the group consisting of PRELP, BCAM, ELN, FBLN5, LAMA3, LAMC1, VCAN, MFAP4, LAMB2, LAMA5, and any combination thereof.

42. The bioink according to claim 37, further comprising at least one additional protein selected from the group consisting of COL1A1, COL1A2, COL3A1, COL5A1, COL5A2, COL16A1, ELN, and any combination thereof.

43. The bioink according to claim 37, further comprising at least one extracellular matrix protein (ECM) protein, or a structural domain thereof, wherein a proportion of the ECM protein, or the structural domain thereof, in the bioink is selected based on a desired tumor grade of a three dimensional (3D) scaffold the 3D scaffold obtainable from the bioink.

44. A three-dimensional (3D) scaffold for cell culturing obtainable from the bioink according to claim 37.

45. The 3D scaffold according to claim 44 obtainable by bioprinting the bioink according to claim 37 by a 3D printer or electrospinning the bioink according to 37.

46. A cell culturing method comprising:
adding cells to a three-dimensional (3D) scaffold according to claim 44; and
culturing the cells in the 3D scaffold.
